SEQUENCE LISTING (2 sequences)

SEQ ID NO: 1 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 2 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKESLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARASLSPATERLYSAVRHVVGQKPTSDRPYIWNDNEQGLDEHIARISADIAAGGVIVQAVQDILPSLH

(19) United States
(12) Patent Application Publication
(10) Pub. No.: US 2023/0183712 A1
(43) Pub. Date: Jun. 15, 2023

(54) METHODS FOR ENGINEERING AMINO ACID AMMONIA LYASE ENZYMES AND ENZYMES THEREBY OBTAINED

(71) Applicant: Trustees of Tufts College, Medford, MA (US)

(72) Inventors: Nikhil Unni Nair, Medford, MA (US); Zachary Mays, Medford, MA (US); Karishma Mohan, Medford, MA (US); Todd Chappell, Malden, MA (US); Vikas Trivedi, Medford, MA (US)

(21) Appl. No.: 17/904,690

(22) PCT Filed: Feb. 18, 2021
(86) PCT No.: PCT/US2021/018491

§ 371 Date: Aug. 19, 2022

Related U.S. Application Data
(60) Provisional application No. 63/003,857, filed on Apr. 1, 2020.
(60) Provisional application No. 62/978,622, filed on Feb. 19, 2020.

(51) Int. Cl. C12N 15/52; C12N 9/88; C12N 15/70; A61K 38/51; A61K 47/60

(52) U.S. Cl. CPC C12N 15/52; C12N 9/88; C12N 15/70; A61K 38/51; A61K 47/60; C12N 2500/32; C12Y 403/01024

(57) ABSTRACT

Disclosed are methods, systems, components, and compositions for engineering enzymes. Particularly disclosed are methods, systems, components, and compositions for engineering phenylalanine ammonia-lyase (PAL) enzymes and isolating variant PAL enzymes with enhanced enzymatic properties. The variant PAL enzymes disclosed herein or obtained by the methods disclosed herein may be utilized for treating diseases or disorders characterized by elevated blood levels of phenylalanine, such as phenylketonuria (PKU).

### CROSS-REFERENCE TO RELATED PATENT APPLICATIONS

The present application claims the benefit of priority under 35 U.S.C. 119(e) to U.S. Provisional Application No. 63/003,857, filed on Apr. 1, 2020, and U.S. Provisional Application No. 62/978,622, filed on Feb. 19, 2020, the contents of which are incorporated herein by reference in their entireties.

### STATEMENT REGARDING FEDERALLY SPONSORED RESEARCH OR DEVELOPMENT

This invention was made with government support under grant HD091798 awarded by the National Institutes of Health. The government has certain rights in the invention.

### BACKGROUND

The field of the invention relates to methods for engineering enzymes. In particular, the field of the invention relates to methods for engineering phenylalanine ammonia-lyase enzymes and isolating phenylalanine ammonia-lyase enzymes with enhanced enzymatic properties via the disclosed methods.

Phenylalanine ammonia lyase (PAL) enzymes are widely found associated with secondary metabolism in plants, bacteria, and fungi. Biocatalytic applications for natural product and fine chemical synthesis has driven the discovery, expression, characterization, and engineering of PAL. More recently, development of PALs for phenylketonuria (PKU) management and cancer therapy has further increased interest in engineering this class of enzymes. Comparative structure and enzyme analysis have informed numerous efforts to improve enzyme stability and alter substrate specificity through rational design. While site-specific mutagenesis has been employed for improving PAL, a more comprehensive mutational landscape has yet to be explored for this class of enzymes. Here, we report development of a directed evolution strategy to engineer PAL enzymes. Central to this approach is a high-throughput enrichment that couples E. coli growth to PAL activity. Applying this method to the PAL from Anabaena variabilis, used in the FDA approved drug Pegvaliase, for PKU treatment, we identified mutations at residues previously unknown as relevant for function that increase higher turnover frequency almost twofold after only a single round of engineering. This work demonstrates the power our technique for ammonia lyase enzyme engineering and is the first example of improving the specific activity of a PAL toward its native substrate.

### SUMMARY

Disclosed are methods, systems, components, and compositions for engineering enzymes. Particularly disclosed are methods, systems, components, and compositions for engineering phenylalanine ammonia-lyase (PAL) enzymes and isolating variant PAL enzymes with enhanced enzymatic properties, such as variant Anabaena variabilis PAL enzymes.

The variant PAL enzymes disclosed herein or obtained by the methods disclosed herein may include amino acid substitutions relative to Anabaena variabilis phenylalanine ammonia-lyase (UniProtKB/Swiss-Prot: Q3M5Z3.1) having the amino acid sequence of SEQ ID NO:1 or a variant having the amino acid sequence of SEQ ID NO:2. Variant PAL enzymes may include one or more amino acid substitutions or combinations of amino acid substitutions at amino acid positions selected from: L4, A7, Q8, S9, K10, F18, G20, N21, S23, N36, N44, L47, T51, 156, G59, I60, S63, 167, N68, A70, I77, M87, T102, N103, L108, M133, I139, M147, 1149, A153, S175, P186, K189, K216, G218, M222, D253, I268, S271, K272, P275, V294, Y304, D306, H307, E308, I339, V344, T345, L349, I350, D353, G360, N400, L406, K413, Y435, F450, N453, N474, V476, R490, K494, K494, T495, A502, R510, T524, D526, D533, and N534. Variant PAL enzymes may include one or more amino acid substitutions or combinations of amino acid substitutions selected from: L4P, L4M, L4P, L4Q, A7S, Q8L, S9G, K10N, F18S, F18C, G20S, N21D, S23G, N36S, N44S, L47P, T51S, I56T, G59D, I60V, S63G, I67V, I67T, I67N, N68D, A70G, I77T, I77V, M87I, M87R, T102A, T102S, T102P, T102D, T102E, T102F, T102H, T102K, T102R, T102S, T102Y, N103S, N103D, N103H, L108Q, L108M, M133I, M133V, I139V, I139T, M147L, M147I, I149F, A153S, S175N, P186A, K189E, K216E, G218A, G218S, M222L, M222V, D253V, I268T, I268S, I268N, I268H, I268V, S271G, K272R, P275S, V294I, Y304F, D306G, D306E, H307N, E308D, I339T, V344I, T345S, L349M, I350V, D353N, G360C, G360N, G360S, N400S, N400D, L406M, L406Q, K413E, Y435F, F450S, N453S, N453A, N453C, N453G, N453L, N453M, N453Q, N474S, V476I, R490S, K494I, K494E, K494N, T495I, A502T, C503S, C503T, R510H, T524A, D526E, D533E, D533N, N534I, and C565S. Preferably, the disclosed variant PAL enzymes have phenylalanine ammonia-lyase activity (EC 4.3.1.24).

The variant PAL enzymes disclosed herein or obtained by the methods disclosed herein may be utilized for treating diseases or disorders characterized by elevated blood levels of phenylalanine. In particular, the variant PAL enzymes disclosed herein or obtained by the methods disclosed herein may be utilized for treating phenylketonuria (PKU).

### BRIEF DESCRIPTION OF THE FIGURES

FIG. 1. Initial study demonstrating growth-rescue of E. coli by PAL activity. (a.) Growth rescue relies on deamination of phenylalanine by PAL to form ammonium (NH₄⁺), a preferred nitrogen source for E. coli. (b.) E. coli cells expressing active AvPAL* (□) in MM^{phe,init} grow faster than wild-type cells (X) or those expressing truncated inactive AvPAL* (0). Cells grown in MM^{full,init} (∘) and MM^{N-,init} (Δ) as controls.

FIG. 2. Optimizing conditions of growth-based PAL selection. E. coli MG1655(DE3)^{ΔendA,ΔrecA} cells expressing AvPAL* (□) or truncated AvPAL* (⋄) were tested for growth in MM^{phe} under different conditions. Growth rates (OD₆₀₀/day) are in the top right corner with optimum conditions being as follows: (a.) Carbon Source, Glucose; (b.) [Phe], 30 mM; (c.) Yeast Extract, 0%; pH, PBS (7.4); (d.) Volume, 1.5 mL. (e.) The final optimized conditions allowed growth recovery in 12 h compared to 3 d previously. (f) While optimizing the growth media, we observed that at phe concentrations >30 mM, final biomass density decreased and lag time increased, suggesting toxicity due to rapid accumulation of tCA. At phe concentrations <30 mM, final biomass densities dropped, and at concentrations ≤7.5 mM, growth rate also slowed, suggesting insufficient nitrogen to sustain growth. (g.) Media supplemented with tCA inhibited the growth of E coli at concentrations ≥1 mM. (h.) AvPAL* expressing cells produce and secrete tCA to ˜1 mM tCA before arresting growth. The subsequent bolus increase in tCA during death phase is likely due to cell lysis. All curves representative of duplicates with less than 10% error.

FIG. 3. Identification of AvPAL* mutants by growth-coupled enrichment. (a.) Purified PAL activity of 15 randomly picked colonies compared to wildtype. (b.) The growth profiles (solid) and tCA production (dotted) in MM^{phe,opt} of select mutants (∘ and Δ). (*** significant compared to wildtype or silent mutants (x), p<0.001).

FIG. 4. Biochemical characterization of PAL mutants. Two mutants showing higher than wildtype activity were characterized to establish (a.) kinetic parameters, (b.) pH optimum, (c.) temperature optimum, (d.) and resistance to protease degradation.

FIG. 5. Growth of E. coli after gene deletions intended to lower basal growth on MM^{phe,init}. (a.-d.) Select aminotransferases with reported promiscuous activity on phenylalanine were deleted in an attempt to reduce the level of basal growth seen by wild-type E. coli on MM^{phe,init}. Each deletion strain showed no changes in growth whether or not expressing AvPAL*. (e.) The ammonia transporter AmtB was also deleted in an attempt to minimize cross-feeding of nitrogen between cells but had no benefit.

FIG. 6. Identification of AvPAL* mutants by growth-coupled enrichment. (a.) The growth profiles of E. coli MG1655^{rph+} cells expressing the AvPAL* mutant library grown in MM^{phe,opt} over three rounds. (b.) SDS-PAGE gels of the lysate of fifteen randomly picked colonies from round 3 of the enrichment. Two gels have been cropped for concision with no image enhancement. (c.) Crude cell lysate activity of the selected mutants normalized to total protein.

FIG. 7. Validating enrichment with a mock library. After transforming a plasmid mix of AvPAL* and sfGFP in 1:10 or 1:1000 ratio, we were able to observe (a.) the loss of fluorescence, and (b.) the enrichment of cells expressing AvPAL* over sfGFP, over rounds of subculturing in MM^{phe} selective media. This was confirmed by (c.) an observed increase in AvPAL* activity on a per cell basis.

FIG. 8. Crystal structure analysis of AvPAL* monomer with active site residues, MIO-adduct, and residues 218 and 222 highlighted. (a.) A top view looking down into the active site. (b.) Side-view of the monomer. (c.) Close up of the wildtype AvPAL* active site with predicted intra-residue hydrogen bonding. (d). Comparison of the wildtype and mutant active sites with residues 218 (left) and 222 (right) highlighted. Mutant residues G218S and M222L have altered intra-residue hydrogen bonding (red, dotted) compared to wildtype (dotted).

FIG. 9. Identified positions in AvPAL having high fitness based on regression analysis.

FIG. 10. Active site of Petroselinum crispum (Pc) PAL from Chem. Rev. 2018, 118, 73-118 and assignment of residues of PcPAL to residues of AvPAL.

FIG. 11. Identified positions in AvPAL having high regression coefficients.

FIG. 12. Focused library design for hyperactive variants via site saturation mutagenesis (SSM)

FIG. 13. Analysis of library prepared as illustrated in FIG. 12. (A) 7×1-55M; (B) 7×1-SSM-Mix; (C) 7×3-SSM; and (D) 7×7-SSM. (E) Vₘₐₓ versus phenylalanine concentration for illustrated variants.

FIG. 14. Table of PAL-variants and calculated Vₘₐₓ, Kₘ, Kᵢ, k_{cat}, k_{cat}/Kₘ, and Fold increase in k_{cat}.

### DETAILED DESCRIPTION

The presently disclosed subject matter is described herein using several definitions, as set forth below and throughout the application.

### Definitions and Terminology

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a gene” or “an oligosaccharide” should be interpreted to mean “one or more genes” and “one or more oligosaccharides,” respectively, unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Polynucleotides and Synthesis Methods

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

The term “promoter” refers to a cis-acting DNA sequence that directs RNA polymerase and other trans-acting transcription factors to initiate RNA transcription from the DNA template that includes the cis-acting DNA sequence.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. As used herein, “expression template” and “transcription template” have the same meaning and are used interchangeably.

In certain exemplary embodiments, vectors such as, for example, expression vectors, containing a nucleic acid encoding one or more polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably.

In certain exemplary embodiments, the recombinant expression vectors comprise a nucleic acid sequence in a form suitable for expression of the nucleic acid sequence in one or more of the methods described herein, which means that the recombinant expression vectors include one or more regulatory sequences which is operatively linked to the nucleic acid sequence to be expressed. The engineered strains disclosed herein may comprise an expression vector which is episomal, such as a plasmid, and/or the engineered strains disclosed herein may comprise an expression vector which is inserted into the genome of the engineered strains.

The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Expression vectors discloses herein by express an exogenous phenylalanine ammonia-lyase (PAL) enzyme.

As utilized herein, a “deletion” means the removal of one or more nucleotides relative to the native polynucleotide sequence. The engineered strains that are disclosed herein may include a deletion in one or more genes. Preferably, a deletion results in a non-functional gene product.

As utilized herein, an “insertion” means the addition of one or more nucleotides to a native polynucleotide sequence. The engineered strains that are disclosed herein may include an insertion in one or more genes. In some embodiments, the engineered strains that are disclosed herein include an insertion of a sequence encoding an exogenous phenylalanine ammonia-lyase (PAL) enzyme. In some embodiments, the engineered strains that are disclosed herein include an insertion in an endogenous gene (i.e., a genomic insertion) which results in a non-functional gene product.

As utilized herein, a “substitution” means replacement of a nucleotide of a native polynucleotide sequence with a nucleotide that is not native to the polynucleotide sequence. The engineered strains that are disclosed herein may include a substitution in one or more genes. In some embodiments, a substitution results in a non-functional gene product, for example, where the substitution introduces a premature stop codon (e.g., TAA, TAG, or TGA) in the coding sequence of the gene product. In some embodiments, the engineered strains that are disclosed herein may include two or more substitutions where the substitutions introduce multiple premature stop codons (e.g., TAATAA, TAGTAG, or TGATGA).

Contemplated herein are polynucleotides that encode polypeptides, including but not limited to polynucleotides that encode phenylalanine ammonia-lyase (PAL), for example, Anabaena variabilis phenylalanine ammonia-lyase (PAL) (SEQ ID NO:1) or variants thereof (SEQ ID NO:2 or other variants thereof as disclosed herein). As contemplated herein, polynucleotides that encode polypeptides (e.g., SEQ ID NO:1, SEQ ID NO:2, or variants thereof as disclosed herein) may be codon-optimized for expression in a particular organism (e.g., E. coli, yeast, or mammalian cells (e.g., human cells)).

Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≥100 amino acids (Garrett & Grisham, Biochemistry, 2^{nd} edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

Reference may be made herein to peptides, polypeptides, proteins and variants or derivatives thereof. Reference amino acid sequences may include, but are not limited to, the amino acid sequence of Anabaena variabilis phenylalanine ammonia-lyase (UniProtKB/Swiss-Prot: Q3M5Z3.1, accessed Feb. 13, 2020) having the amino acid sequence of SEQ ID NO:1:

| | |
|---|---|
| 1 | MKTLSQAQSK TSSQQFSFTG NSSANVIIGN |
| | QKLTINDVAR VARNGTLVSL |
| 51 | TNNTDILQGI QASCDYINNA VESGEPIYGV |
| | TSGFGGMANV AISREQASEL |
| 101 | QTNLVWFLKT GAGNKLPLAD VRAAMLLRAN |
| | SHMRGASGIR LELIKRMEIF |
| 151 | LNAGVTPYVY EFGSIGASGD LVPLSYITGS |
| | LIGLDPSFKV DFNGKEMDAP |
| 201 | TALRQLNLSP LTLLPKEGLA MMNGTSVMTG |
| | IAANCVYDTQ ILTAIAMGVH |
| 251 | ALDIQALNGT NQSFHPFIHN SKPHPGQLWA |
| | ADQMISLLAN SQLVRDELDG |
| 301 | KHDYRDHELI QDRYSLRCLP QYLGPIVDGI |
| | SQIAKQIEIE INSVTDNPLI |
| 351 | DVDNQASYHG GNFLGQYVGM GMDHLRYYIG |
| | LLAKHLDVQI ALLASPEFSN |
| 401 | GLPPSLLGNR ERKVNMGLKG LQICGNSIMP |
| | LLTFYGNSIA DRFPTHAEQF |
| 451 | NQNINSQGYT SATLARRSVD IFQNYVAIAL |
| | MFGVQAVDLR TYKKTGHYDA |
| 501 | RACLSPATER LYSAVRHWG QKPTSDRPYI |
| | WNDNEQGLDE HIARISADIA |
| 551 | AGGVIVQAVQ DILPCLH |

Reference amino acid sequences may include, but are not limited to, the amino acid sequence of variants of Anabaena variabilis phenylalanine ammonia-lyase having amino acid substitutions C503S and C565S, phenylalanine ammonia lyase activity (EC 4.3.1.24) and the amino acid sequence of SEQ ID NO:2:

| | |
|---|---|
| 1 | MKTLSQAQSK TSSQQFSFTG NSSANVIIGN |
| | QKLTINDVAR VARNGTLVSL |
| 51 | TNNTDILQGI QASCDYINNA VESGEPIYGV |
| | TSGFGGMANV AISREQASEL |
| 101 | QTNLVWFLKT GAGNKLPLAD VRAAMLLRAN |
| | SHMRGASGIR LELIKRMEIF |
| 151 | LNAGVTPYVY EFGSIGASGD LVPLSYITGS |
| | LIGLDPSFKV DFNGKEMDAP |
| 201 | TALRQLNLSP LTLLPKESLA MMNGTSVMTG |
| | IAANCVYDTQ ILTAIAMGVH |
| 251 | ALDIQALNGT NQSFHPFIHN SKPHPGQLWA |
| | ADQMISLLAN SQLVRDELDG |
| 301 | KHDYRDHELI QDRYSLRCLP QYLGPIVDGI |
| | SQIAKQIEIE INSVTDNPLI |
| 351 | DVDNQASYHG GNFLGQYVGM GMDHLRYYIG |
| | LLAKHLDVQI ALLASPEFSN |
| 401 | GLPPSLLGNR ERKVNMGLKG LQICGNSIMP |
| | LLTFYGNSIA DRFPTHAEQF |
| 451 | NQNINSQGYT SATLARRSVD IFQNYVAIAL |
| | MFGVQAVDLR TYKKTGHYDA |
| 501 | RASLSPATER LYSAVRHVVG QKPTSDRPYI |
| | WNDNEQGLDE HIARISADIA |
| 551 | AGGVIVQAVQ DILPSLH |

In some embodiments, variants or derivatives as contemplated herein may have an amino acid sequence that includes conservative amino acid substitutions or non-conservative amino acid substitutions relative to a reference amino acid sequence. For example, a variant or derivative peptide, polypeptide, or protein as contemplated herein may include conservative amino acid substitutions and/or non-conservative amino acid substitutions relative to a reference peptide, polypeptide, or protein. “Conservative amino acid substitutions” are those substitutions that are predicted to interfere least with the properties of the reference peptide, polypeptide, or protein, and “non-conservative amino acid substitution” are those substitution that are predicted to interfere most with the properties of the reference peptide, polypeptide, or protein. In other words, conservative amino acid substitutions substantially conserve the structure and the function of the reference peptide, polypeptide, or protein, whereas non-conservative amino acid substitutions do not conserve the structure and the function of the reference peptide, polypeptide, or protein. The following table provides a list of exemplary conservative amino acid substitutions.

TABLE 1

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally disrupt: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain.

In some embodiments of the variant PAL enzymes disclosed herein, the variant PAL enzymes may include one or more amino acid substitutions or combinations of amino acid substitutions at amino acid positions of SEQ ID NO:1 or SEQ ID NO:2 selected from: L4, A7, Q8, S9, K10, F18, G20, N21, S23, N36, N44, L47, T51, 156, G59, I60, S63, 167, N68, A70, I77, M87, T102, N103, L108, M133, I139, M147, I149, A153, S175, P186, K189, K216, G218, M222, D253, I268, S271, K272, P275, V294, Y304, D306, H307, E308, I339, V344, T345, L349, I350, D353, G360, N400, L406, K413, Y435, F450, N453, N474, V476, R490, K494, K494, T495, A502, R510, T524, D526, D533, and N534. Preferably, the disclosed variant PAL enzymes have phenylalanine ammonia-lyase activity (EC 4.3.1.24), which may be enhanced activity relative to the wild-type PAL enzyme.

In some embodiments of the variant PAL enzymes disclosed herein, the variant PAL enzymes may include one or more amino acid substitutions or combinations of amino acid substitutions of SEQ ID NO:1 selected from: L4P, L4M, L4P, L4Q, A7S, Q8L, S9G, K10N, F185, F18C, G20S, N21D, S23G, N36S, N44S, L47P, T51S, I56T, G59D, I60V, S63G, I67V, I67T, I67N, N68D, A70G, I77T, I77V, M87I, M87R, T102A, T102S, T102P, T102D, T102E, T102F, T102H, T102K, T102R, T102S, T102Y, N103S, N103D, N103H, L108Q, L108M, M133I, M133V, I139V, I139T, M147L, M147I, I149F, A1535, S175N, P186A, K189E, K216E, G218A, G2185, M222L, M222V, D253V, I268T, I268S, I268N, I268H, I268V, S271G, K272R, P275S, V294I, Y304F, D306G, D306E, H307N, E308D, I339T, V344I, T345S, L349M, I350V, D353N, G360C, G360N, G360S, N400S, N400D, L406M, L406Q, K413E, Y435F, F450S, N453S, N453A, N453C, N453G, N453L, N453M, N453Q, N474S, V476I, R490S, K494I, K494E, K494N, 14951, A502T, C503S, C503T, R510H, T524A, D526E, D533E, D533N, N534I, and C565S. Preferably, the disclosed variant PAL enzymes have phenylalanine ammonia-lyase activity (EC 4.3.1.24). Preferably, the disclosed variant PAL enzymes have phenylalanine ammonia-lyase activity (EC 4.3.1.24), which may be enhanced activity relative to the wild-type PAL enzyme.

Variants or derivatives comprising deletions relative to a reference amino acid sequence of peptide, polypeptide, or protein are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation of a reference polypeptide or a 5′-terminal or 3′-terminal truncation of a reference polynucleotide).

Variants or derivatives comprising a fragment of a reference amino acid sequence of a peptide, polypeptide, or protein are contemplated herein. A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full length polypeptide.

Variants or derivatives comprising insertions or additions relative to a reference amino acid sequence of a peptide, polypeptide, or protein are contemplated herein. The words “insertion” and “addition” refer to changes in an amino acid or sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues.

Fusion proteins also are contemplated herein. A “fusion protein” refers to a protein formed by the fusion of at least one peptide, polypeptide, or protein or variant or derivative thereof as disclosed herein to at least one heterologous protein peptide, polypeptide, or protein (or fragment or variant or derivative thereof). The heterologous protein(s) may be fused at the N-terminus, the C-terminus, or both termini of the peptides or variants or derivatives thereof.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number (e.g., any of SEQ ID NO:1 or SEQ ID NO:2), or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” or “derivative” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” or “derivative” may have substantially the same functional activity as a reference polypeptide (e.g., glycosylase activity or other activity). “Substantially isolated or purified” amino acid sequences are contemplated herein. The term “substantially isolated or purified” refers to amino acid sequences that are removed from their natural environment, and are at least 60% free, preferably at least 75% free, and more preferably at least 90% free, even more preferably at least 95% free from other components with which they are naturally associated. Variant or derivative polypeptides as contemplated herein may include variant or derivative polypeptides of any of SEQ ID NO:1 or SEQ ID NO:2).

Reactions and Components

The term “reaction mixture,” as used herein, refers to a solution containing reagents necessary to carry out a given reaction. A reaction mixture is referred to as complete if it contains all components necessary to perform the reaction. Components for a reaction mixture may be stored together in a single container or separately in separate containers, each containing one or more of the total components. Components may be packaged separately for commercialization and useful commercial kits may contain one or more of the reaction components for a reaction mixture.

The steps of the methods described herein can be performed in any suitable order unless otherwise indicated herein or otherwise clearly contradicted by context. The steps may be repeated or reiterated any number of times to achieve a desired goal unless otherwise indicated herein or otherwise clearly contradicted by context.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The disclosed components may be in crude form and/or may be at least partially isolated and/or purified. As used herein, the term “isolated or purified” may refer to components that are removed from their natural environment and/or media, and are at least 60% free, preferably at least 75% free, and more preferably at least 90% free, even more preferably at least 95% free from other components with which they are naturally associated and/or media.

Pharmaceutical Compositions

The compositions disclosed herein may include pharmaceutical compositions comprising the presently disclosed variants and formulated for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”) such as phenylketonuria (PKU)).

Miscellaneous

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Methods for Engineering Amino Acid Ammonia Lysase Enzymes and Enzymes Thereby Obtained

Disclosed are methods, systems, components, and compositions for engineering enzymes. Particularly disclosed are methods, systems, components, and compositions for engineering phenylalanine ammonia-lyase (PAL) enzymes and isolating variant PAL enzymes with enhanced enzymatic properties, such as variant Anabaena variabilis PAL enzymes. The variant PAL enzymes disclosed herein or obtained by the methods disclosed herein may be utilized for treating diseases or disorders characterized by elevated blood levels of phenylalanine, such as phenylketonuria (PKU).

In some embodiments, the disclosed subject matter relates to variants of PAL enzymes. In particular, the inventors have identified variants of Anabaena variabilis PAL enzyme (SEQ ID NO:1). In some embodiments, the variants comprise amino acid substitutions that comprise one or more of the following identified amino acid substitutions which were identified by the inventors: L4P, L4M, L4P, L4Q, A7S, Q8L, S9G, K10N, F185, F18C, G20S, N21D, S23G, N36S, N44S, L47P, T51S, I56T, G59D, I60V, S63G, I67V, I67T, I67N, N68D, A70G, I77T, I77V, M87I, M87R, T102A, T102S, T102P, T102D, T102E, T102F, T102H, T102K, T102R, T102S, T102Y, N103S, N103D, N103H, L108Q, L108M, M133I, M133V, I139V, I139T, M147L, M147I, I149F, A1535, S175N, P186A, K189E, K216E, G218A, G2185, M222L, M222V, D253V, I268T, I268S, I268N, I268H, I268V, S271G, K272R, P275S, V294I, Y304F, D306G, D306E, H307N, E308D, I339T, V344I, T345S, L349M, I350V, D353N, G360C, G360N, G360S, N400S, N400D, L406M, L406Q, K413E, Y435F, F450S, N453S, N453A, N453C, N453G, N453L, N453M, N453Q, N474S, V476I, R490S, K494I, K494E, K494N, T495I, A502T, C503S, C503T, R510H, T524A, D526E, D533E, D533N, N534I, and C565S. In some embodiments, the variants comprise a combination of amino acid substitutions that comprise: (i) C503S and C565S (SEQ ID NO:2); and (ii) one or more of L4P, L4M, L4P, L4Q, A7S, Q8L, S9G, K10N, F18S, F18C, G20S, N21D, S23G, N36S, N44S, L47P, T51S, I56T, G59D, I60V, S63G, I67V, I67T, I67N, N68D, A70G, I77T, I77V, M87I, M87R, T102A, T102S, T102P, T102D, T102E, T102F, T102H, T102K, T102R, T102S, T102Y, N103S, N103D, N103H, L108Q, L108M, M133I, M133V, I139V, I139T, M147L, M147I, I149F, A153S, S175N, P186A, K189E, K216E, G218A, G218S, M222L, M222V, D253V, I268T, I268S, I268N, I268H, I268V, S271G, K272R, P275S, V294I, Y304F, D306G, D306E, H307N, E308D, I339T, V344I, T345S, L349M, I350V, D353N, G360C, G360N, G360S, N400S, N400D, L406M, L406Q, K413E, Y435F, F450S, N453S, N453A, N453C, N453G, N453L, N453M, N453Q, N474S, V476I, R490S, K494I, K494E, K494N, 14951, A502T, R510H, T524A, D526E, D533E, D533N, and N534I. In some embodiments, the variants comprise an amino acid substitution at one or more of amino acid position G360 and N453, where the variant comprises other than a G residue at amino acid position 360 and/or the variant comprises other than an N residue at amino acid position 453.

In some embodiments, the variant comprises one or more amino acid substitutions at an amino acid position listed in the following table or the variant comprises one or more of the specific amino acid substitutions listed in the following table.

| Residue | Substitution | Residue | Substitution | Residue | Substitution |
|---|---|---|---|---|---|
| L4 | M, P⁺, Q | N103 | D, S, H | V344 | I |
| A7 | S | L108 | M, Q | T345 | S |
| Q8 | L | M133 | I, V | L349 | M |
| S9 | G | I139 | V, T | I350 | V |
| K10 | N | M147 | I, L | D353 | N |
| F18 | S, C | I149 | F | G360* | C⁺, N, S |
| G20 | S | A153 | S | N400 | S, D |
| N21 | D | S175 | N | L406 | M, Q |
| S23 | G | P186 | A | K413 | E |
| N36 | S | K189 | E | Y435 | F |
| N44 | S | F192 | Y | F443 | Y |
| L47 | P | T212 | A | F450 | S |
| T51 | S | L213 | P | N453* | S⁺, A⁺, C, |
| | | | | | G⁺, L, M, Q |
| I56 | T | K216 | E | N474 | S |
| G59 | D | G218* | A⁺, S⁺ | V476 | I |
| I60 | V | M222* | L⁺, V | R490 | S |
| S63 | G | D253 | V | K494 | I, E, N |
| I67 | V, T, N | I268* | S, T, N⁺, | T495 | I |
| | | | H⁺, V⁺ | | |
| N68 | D | S271 | G | A502 | T |
| A70 | G | K272 | R | S503 | T |
| I77 | T, V | P275 | S | R510 | H |
| M87 | I, R | V294 | I | T524 | A |
| V90 | L, F | Y304 | F | D526 | E |
| A91 | V | D306* | G⁺, E | D533 | E, N |
| Q96 | L | H307 | N | N534 | I |
| S98 | T, C, N | E308 | D | | |
| T102* | A⁺, S⁺, P⁺, | I339 | T | | |
| | D, E⁺, F, H, | | | | |
| | K⁺, R⁺, Y | | | | |

*Top seven sites for identified substitutions
⁺Identified substitutions present in variants observed to have high activity

The variants disclosed herein preferably have PAL activity (EC 4.3.1.24). In some embodiments, the disclosed variants have a k_{cat} with respect to producing trans-cinnamic acid from phenylalanine that is higher than wild-type Anabaena variabilis PAL enzyme (SEQ ID NO:1) or a variant of Anabaena variabilis PAL enzyme comprising amino acid substitutions that comprise C503S and C565S (SEQ ID NO:2).

Conjugates also are contemplated herein. In some embodiments, the disclosed conjugated comprise a variant PAL enzyme as disclosed herein conjugated to a moiety which increases the variant PAL enzyme's half-life when administered to a subject in need thereof. In some embodiments, the disclosed conjugates comprise a variant PAL enzyme as disclosed herein conjugated to a polyethylene glycol (PEG) polymer.

Pharmaceutical compositions also are contemplated herein. In some embodiments, the disclosed pharmaceutical compositions comprise: (i) a variant PAL enzyme as contemplated herein; and (ii) a suitable pharmaceutical carrier. In other embodiments, the disclosed pharmaceutical compositions comprise: (i) a conjugate of a variant PAL enzyme as contemplated herein (e.g., a conjugate to PEG polymer); and (ii) a suitable pharmaceutical carrier.

Also contemplated herein are polynucleotides, such as polynucleotides encoding the variant PAL enzymes as contemplated herein. The polynucleotides may be codon-optimized. In some embodiments, the polynucleotide is codon-optimized for expression of the variant PAL enzyme in Escherichia coli. In other embodiments, the polynucleotide is codon-optimized for expression of the variant PAL enzyme in human cells. The disclosed polynucleotides may be present in vectors as known in the art (e.g., plasmid vectors).

Expression vectors also are contemplated herein. In some embodiments, the expression vectors are configured for expressing a variant PAL enzyme as contemplated herein, for example, wherein the expression vectors comprise a promoter operably linked to a polynucleotide encoding the variant PAL enzyme.

The disclosed polynucleotides and/or vectors may be present in a cell that has been modified via introducing the polynucleotide and/or vectors to the modified cell, for example, via transformation. Suitable cells may include but are not limited to Escherichia coli and/or human cells.

Also disclosed are methods for preparing the variant PAL enzymes disclosed herein. In some embodiments, the disclosed methods comprise culturing in culture media a modified cell that has been modified via introduction of a polynucleotide and/or vector that encodes and expresses the variant PAL enzyme and isolating the variant PAL enzyme from the modified cell and/or culture media.

Methods of treatment also are contemplated herein, such as methods for treating a disease or disorder in a subject in need thereof, wherein the disease or disorder is characterized by elevated blood levels of phenylalanine. The methods may comprise administering to the subject one or more of the following agents: (i) a variant PAL enzyme as contemplated herein; (ii) a conjugate comprising a variant PAL enzyme as contemplated herein (e.g., conjugated to PEG polymer); (iii) a pharmaceutical composition comprising a variant PAL enzyme or a conjugate thereof; (iv) a polynucleotide and/or vector encoding and/or expressing a variant PAL enzyme as contemplated herein; and/or (v) a modified cell that expresses a variant PAL enzyme as contemplated herein (e.g., as a probiotic). The agent may be administered by any suitable method, including but not limited to subcutaneously. Suitable diseases and disorders treated by the disclosed methods may include, but are not limited to phenylketonuria (PKU).

Also disclosed herein are methods for obtaining a variant of a PAL enzyme. The disclosed methods may comprise one or more of the following steps: (i) transforming cells that cannot utilize phenylalanine to obtain nitrogen with a library of expression vectors that encode and/or express variants of the PAL enzymes in the transformed cells; (ii) culturing the transformed cells in a minimal media that is supplemented with phenylalanine; (iii) selecting transformed cells that grow in the minimal media that is supplemented with phenylalanine; (iv) determining the sequence of the encoded phenylalanine ammonia-lyase of transformed cells that grow in the minimal media that is supplemented with phenylalanine.

In the disclosed methods for obtaining a variant of a PAL enzyme, suitable variant PAL enzymes for the disclosed methods may include, but are not limited to variants of Anabaena variabilis PAL enzyme. Suitable transformed cells may include but are not limited to transformed Escherichia coli where preferably the expression vectors have been codon-optimized for expression of the variant of PAL enzyme in Escherichia coli.

In the disclosed methods for obtaining a variant of a PAL enzyme, the minimal media is supplemented with phenylalanine. Preferably the minimal media is supplemented with phenylalanine to a concentration of about 20-40 mM.

The minimal media may be optimized for growth of the transformed cells by further supplementing and/or modifying the minimal media. In some embodiments, the minimal media is supplemented with glucose, optionally at a concentration of 0.1-0.3 (v/v)). In further embodiments, the minimal media does not comprise glycerol.

In the disclosed methods for obtaining a variant of a PAL enzyme, the transformed cells may be subculturing one or more times. For example, the transformed cells may be removed from the culture media after the culture media reaches an OD₆₀₀ of at least about 1-2 (e.g., when the transformed cells are still in growth phase) and placing the transformed cells into fresh minimal media supplemented with phenylalanine. This optional subculturing step may be performed one or more times. Optionally, the subculturing step is performed to remove the transformed cells from the culture when the culture comprises excess tCA, for example, when the culture comprises at least 0.5, 1.0, 1.5, or 2.0 mM tCA.

### Illustrative Embodiments

The following embodiments are illustrative and should not be interpreted to limit the scope of the claimed subject matter.

Embodiment 1. A variant phenylalanine ammonia-lyase (PAL) enzyme of Anabaena variabilis (SEQ ID NO:1) comprising amino acid substitutions that comprise one or more of: L4P, L4M, L4P, L4Q, A7S, Q8L, S9G, K10N, F185, F18C, G20S, N21D, S23G, N36S, N44S, L47P, T51S, I56T, G59D, I60V, S63G, I67V, I67T, I67N, N68D, A70G, I77T, I77V, M87I, M87R, T102A, T102S, T102P, T102D, T102E, T102F, T102H, T102K, T102R, T102S, T102Y, N103S, N103D, N103H, L108Q, L108M, M133I, M133V, I139V, I139T, M147L, M147I, I149F, A1535, S175N, P186A, K189E, K216E, G218A, G2185, M222L, M222V, D253V, I268T, I268S, I268N, I268H, I268V, S271G, K272R, P275S, V294I, Y304F, D306G, D306E, H307N, E308D, I339T, V344I, T345S, L349M, I350V, D353N, G360C, G360N, G360S, N400S, N400D, L406M, L406Q, K413E, Y435F, F450S, N453S, N453A, N453C, N453G, N453L, N453M, N453Q, N474S, V476I, R490S, K494I, K494E, K494N, T495I, A502T, C503S, C503T, R510H, T524A, D526E, D533E, D533N, N534I, and C565S; optionally a variant phenylalanine ammonia-lyase (PAL) enzyme of Anabaena variabilis (SEQ ID NO:1) comprising amino acid substitutions that comprise: (i) C503S and C565S; and/or one or more of (ii) L4P, L4M, L4P, L4Q, A7S, Q8L, S9G, K10N, F185, F18C, G20S, N21D, S23G, N36S, N44S, L47P, T51S, I56T, G59D, I60V, S63G, I67V, I67T, I67N, N68D, A70G, I77T, I77V, M87I, M87R, T102A, T102S, T102P, T102D, T102E, T102F, T102H, T102K, T102R, T102S, T102Y, N103S, N103D, N103H, L108Q, L108M, M133I, M133V, I139V, I139T, M147L, M147I, I149F, A1535, S175N, P186A, K189E, K216E, G218A, G2185, M222L, M222V, D253V, I268T, I268S, I268N, I268H, I268V, S271G, K272R, P275S, V294I, Y304F, D306G, D306E, H307N, E308D, I339T, V344I, T345S, L349M, I350V, D353N, G360C, G360N, G360S, N400S, N400D, L406M, L406Q, K413E, Y435F, F450S, N453S, N453A, N453C, N453G, N453L, N453M, N453Q, N474S, V476I, R490S, K494I, K494E, K494N, T495I, A502T, R510H, T524A, D526E, D533E, D533N, and N534I.

Embodiment 2. The variant PAL enzyme of embodiment 1, wherein the variant has a k_{cat} with respect to producing trans-cinnamic acid from phenylalanine that is higher than wild-type Anabaena variabilis (SEQ ID NO:1) or a variant PAL enzyme of Anabaena variabilis (SEQ ID NO:1) comprising amino acid substitutions that comprise C503S and C565S (SEQ ID NO:2); and/or the variant exhibit higher stability (e.g., a longer half-life) at a temperature of at least 37° C., 45° C., 50° C., 55° C., 60° C., or 65° C. than wild-type Anabaena variabilis (SEQ ID NO:1) or a variant PAL enzyme of Anabaena variabilis (SEQ ID NO:1) comprising amino acid substitutions that comprise C503S and C565S (SEQ ID NO:2).

Embodiment 3. A conjugate comprising the variant PAL enzyme of embodiment 1 or 2 conjugated to a polyethylene glycol (PEG) polymer.

Embodiment 4. A pharmaceutical composition comprising; (i) the variant PAL enzyme of embodiments 1 or 2 or the conjugate of embodiment 3 and (ii) a suitable pharmaceutical carrier.

Embodiment 5. A polynucleotide encoding the variant PAL enzyme of embodiment 1 or embodiment 2.

Embodiment 6. The polynucleotide of embodiment 5, wherein the polynucleotide is codon-optimized for expression of the variant PAL enzyme in Escherichia coli.

Embodiment 7. The polynucleotide of embodiment 5, wherein the polynucleotide is codon-optimized for expression of the variant PAL enzyme in human cells.

Embodiment 8. An expression vector for expressing the variant PAL enzyme of embodiment 1 or 2, optionally comprising a promoter operably linked to the polynucleotide of any of embodiments 5-7.

Embodiment 9. A modified cell comprising the variant PAL enzyme of embodiment 1 or 2; the polynucleotide of any of embodiments 5-7; and/or the expression vector of embodiment 8, optionally wherein the cell is a modified Escherichia coli cell or modified human cell.

Embodiment 10. A method for preparing the variant of embodiment 1 or 2, the method comprising culturing the modified cell of embodiment 9 in culture media to express the variant PAL enzyme and isolating the variant PAL enzyme from the modified cell and/or culture media.

Embodiment 11. A method for treating a disease or disorder in a subject in need thereof, wherein the disease or disorder is characterized by elevated blood levels of phenylalanine, the method comprising administering to the subject the variant PAL enzyme of embodiment 1 or 2; the conjugate of embodiment 3; the pharmaceutical composition of embodiment 4; the polynucleotide of any of embodiments 5-7; the expression vector of embodiment 8; and/or the modified cell of embodiment 9 or 10.

Embodiment 12. The method of embodiment 11, wherein the subject is administered the variant PAL enzyme of embodiment 1 or 2 or the conjugate of embodiment 3 subcutaneously.

Embodiment 13. The method of embodiment 11, wherein the disease or disorder is phenylketonuria (PKU).

Embodiment 14. A method for obtaining a variant of a PAL enzyme; the method comprising one or more of the following steps: (i) transforming cells that cannot utilize phenylalanine to obtain nitrogen with a library of expression vectors that encode and express variants of the PAL enzymes in the transformed cells; (ii) culturing the transformed cells in a minimal media that is supplemented with phenylalanine; (iii) selecting transformed cells that grow in the minimal media that is supplemented with phenylalanine; (iv) determining the sequence of the encoded PAL enzyme of a transformed cell that grows in the minimal media that is supplemented with phenylalanine.

Embodiment 15. The method of embodiment 14, wherein the variant of PAL enzyme is a variant of Anabaena variabilis PAL enzyme.

Embodiment 16. The method of embodiment 14 or 15, wherein the transformed cells are transformed Escherichia coli and the expression vectors have been codon-optimized for expression of the variant of the PAL enzyme in Escherichia coli.

Embodiment 17. The method of any of embodiments 14-16, wherein the minimal media is supplemented with glucose, optionally at a concentration of 0.1-0.3 (v/v)).

Embodiment 18. The method of any of embodiments 14-17, wherein the minimal media does not comprise glycerol.

Embodiment 19. The method of any of embodiments 14-18, wherein the minimal media is supplemented with phenylalanine at a concentration of 20-40 mM.

Embodiment 20. The method of any of embodiments 14-19, wherein culturing comprises subculturing the transformed cells by removing the cells from the culture media after the culture media reaches an OD₆₀₀ of at least about 1.8-2.2 and placing the transformed cells into fresh minimal media supplemented with phenylalanine.

### Examples

The following Examples are illustrative and are not intended to limit the scope of the claimed subject matter.

Title—Directed Evolution of Anabaena variabilis Phenylalanine Ammonia-Lyase (PAL) Identifies Mutants with Enhanced Activities

Reference is made to the manuscript Mays et al., “Directed Evolution of Anabaena variabilis phenylalanine ammonia-lyase (PAL) identifies mutants with enhanced activities,” Chem Commun (Camb). 2020 May 14; 56(39):5255-5258, the content of which is incorporated herein by reference in its entirety.

There is broad interest in engineering phenylalanine ammonia-lyase (PAL) for its biocatalytic applications in industry and medicine. While site-specific mutagenesis has been employed to improve PAL stability or substrate specificity, combinatorial techniques are poorly explored. Here, we report development of a directed evolution technique to engineer PAL enzymes. Central to this approach is a high-throughput enrichment that couples E. coli growth to PAL activity. Starting with the PAL used in the formulation of pegvaliase for PKU therapy, we report previously unidentified mutations that increase turnover frequency almost twofold after only a single round of engineering.

The ammonia lyase (AL; EC 4.3.1.x) class and aminomutase (AM; 5.4.3.x) class of enzymes have been the focus of decades of research and development for industrial and biomedical applications. Their prosthetic group, 4-methylideneimidazole-5-one (MIO), either catalyzes the transformation of an L-α-amino acid into the α,β-unsaturated carboxylic acid counterpart via the non-oxidative elimination of ammonia or into the spatially isometric β-amino acid, respectively¹. Hence, application of MIO-enzymes in both directions has yielded intermediates for pharmaceuticals^{2,3}, agrochemicals⁴⁻⁶, polymers⁷⁻⁹, and flavonoids^{2,10-12}.

Phenylalanine ammonia lyase (PAL) has been of great interest as a treatment for the genetic disease phenylketonuria (PKU). Daily subcutaneous injection of a purified and PEGylated recombinant PAL from Anabaena variabilis (PEG-rAvPAL; Palynziq®, BioMarin Pharmaceutical Inc.) was approved by the US FDA in 2018 as an enzyme substitution therapy for PKU¹³. Concurrently, an orally administered engineered PAL-expressing probiotic Escherichia coli Nissle 1917 is currently under investigation by Synlogic Inc¹⁴. Other formulations of this enzyme are also being explored as therapeutics' as well as for the production of low phenylalanine (phe) protein dietary supplementation for PKU¹⁷⁻²⁰ and cancer²¹⁻²⁴ patients.

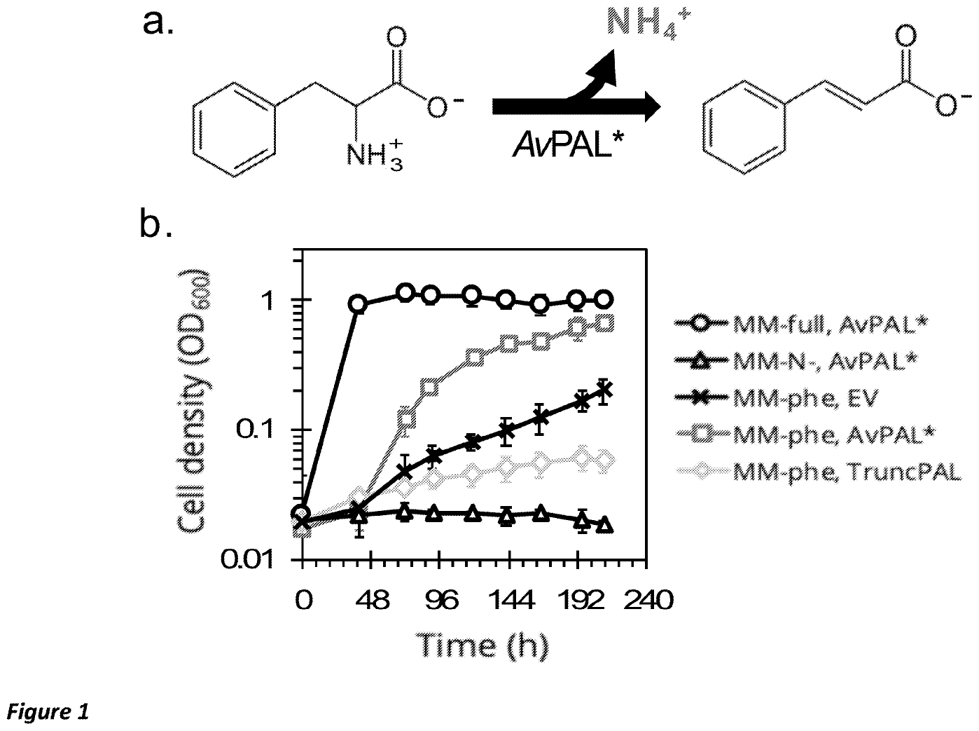

This significant interest has resulted in various efforts to improve enzyme properties. Structural and sequence homology between aromatic ALs and AMs has fuelled rational engineering efforts to alter or improve stability²⁵⁻²⁷, substrate specificity²⁸, and enantioselectivity²⁹. However, application of combinatorial approaches that leverage evolutionary selection to search large sequence spaces for improved properties³⁰ has not been well-explored for this class of enzymes^{31,32}. Here, we developed a growth-based high-throughput enrichment scheme and screened a mutagenized PAL library to identify variants with improved kinetic properties. Core to this enrichment is the growth rescue of E. coli by PAL in minimal medium with phe, which cannot be used as the sole nitrogen source by K-12 strains³³. Consequently, E. coli can only grow if PAL actively deaminates phe to release ammonium, a highly preferred nitrogen-source (FIG. 1a). We executed our directed evolution technique using the A. variabilis PAL (DM-rAvPAL/AvPAL*)²⁰, because of its clinical significance, and identified mutants with improved catalytic properties. The mutations identified here have not been previously reported as important for PAL catalytic activity, demonstrating the advantage of our approach for scanning unexplored sequence space over previous efforts.

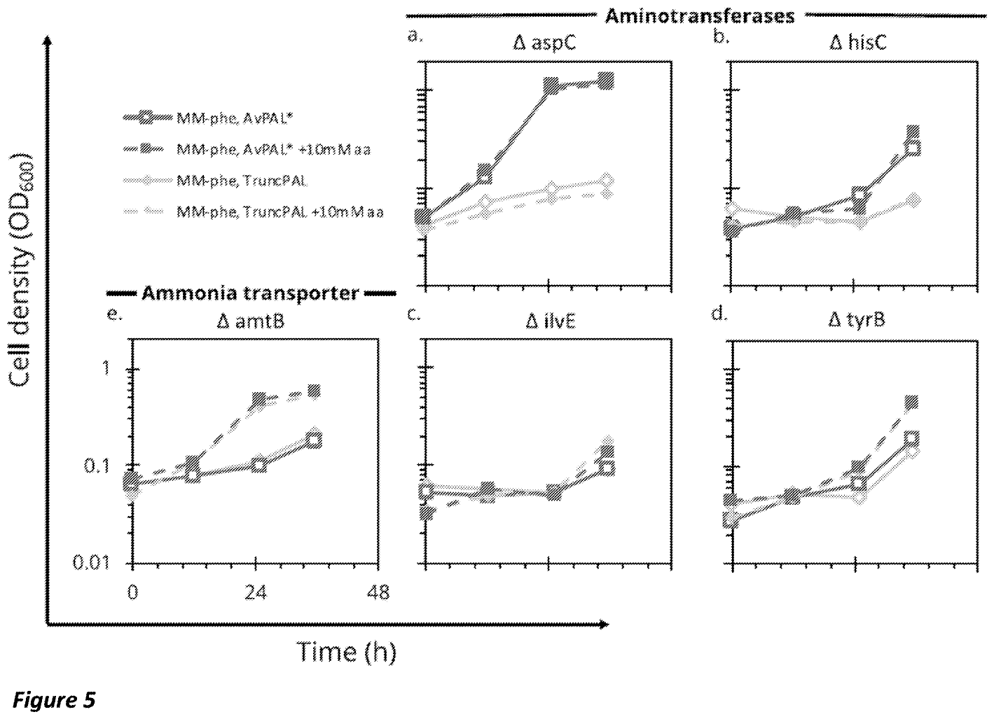

Initial growth studies demonstrated that AvPAL* could rescue growth of E. coli in phe selective minimal media (MM^{phe,init}) with a ˜70% biomass yield relative to complete minimal media (MM^{full,init}), demonstrating coupling between growth and enzyme activity (FIG. 1b). However, controls strains had unexpected, albeit slow, growth. Overexpression of noncatalytic proteins such as green fluorescent protein (sfGFP) or a truncated AvPAL* (TruncPAL) decreased background growth (FIG. 1b) but still adversely affected the dynamic range to reliably select for highly active PAL over inactive mutants or other suppressors, if left unoptimized. Phenylalanine metabolism under austere conditions, viz nitrogen starvation, has not been well studied, and transaminases (AspC, IlvE, TyrB, HisC) may have unreported promiscuous activity on phe. Unfortunately, we observed no difference in the basal growth of E. coli in MM^{phe,init} after deleting each transaminase (FIG. 5) suggesting no single gene was responsible for basal growth.

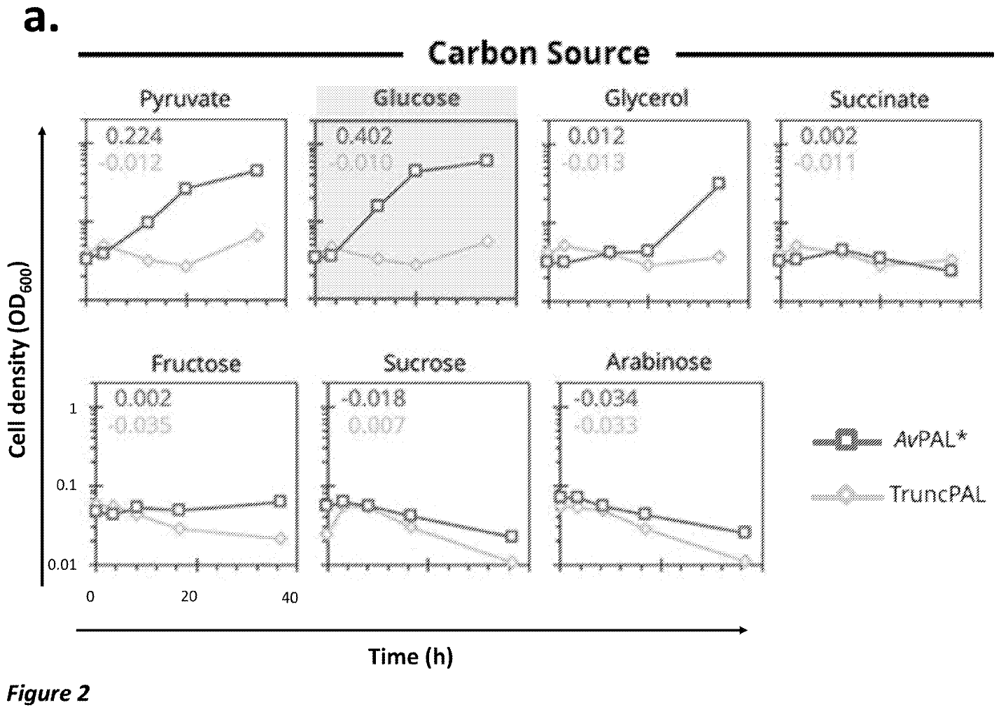

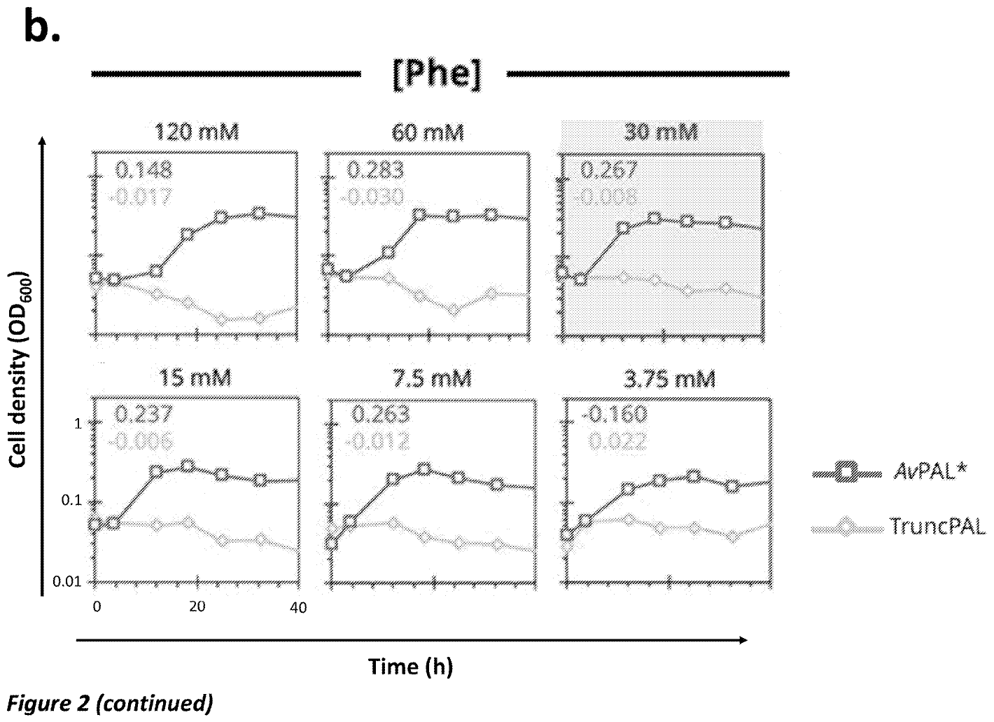

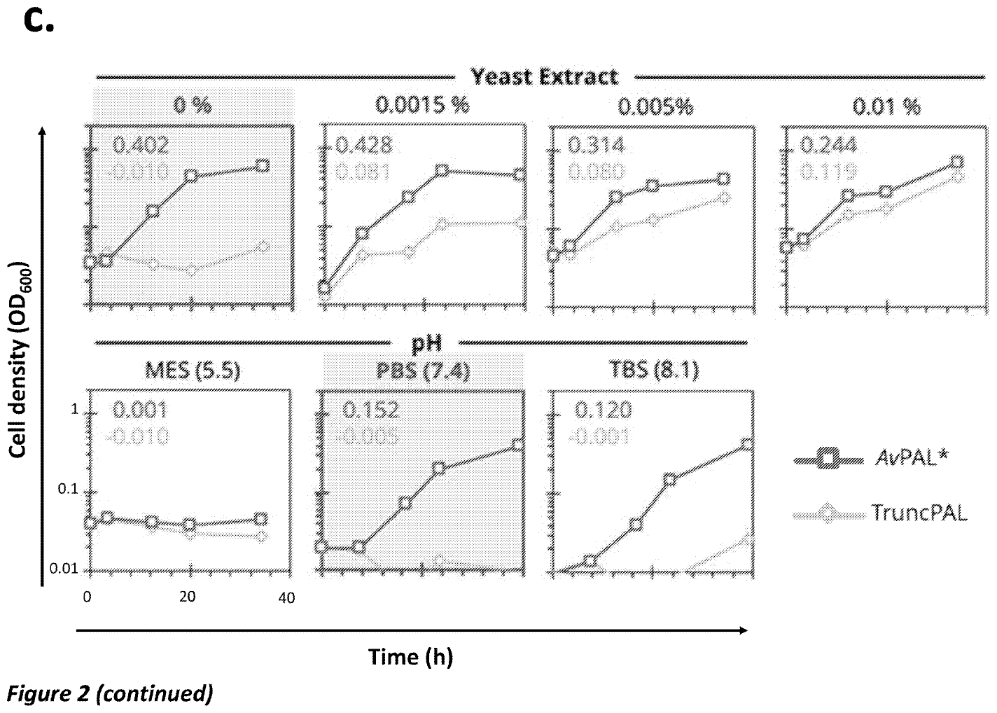

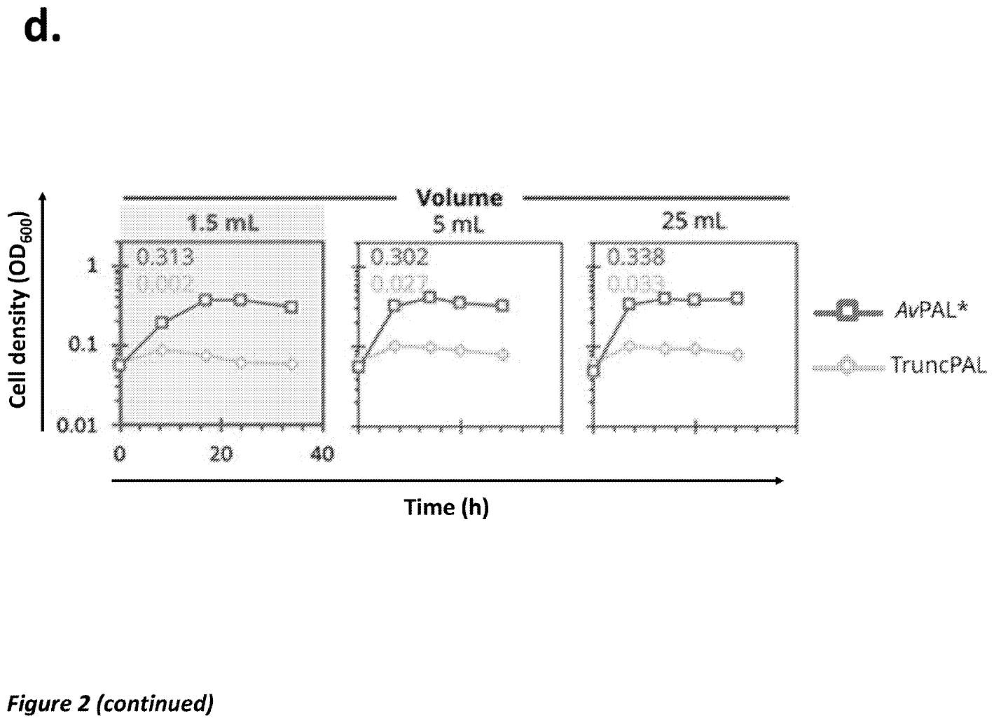

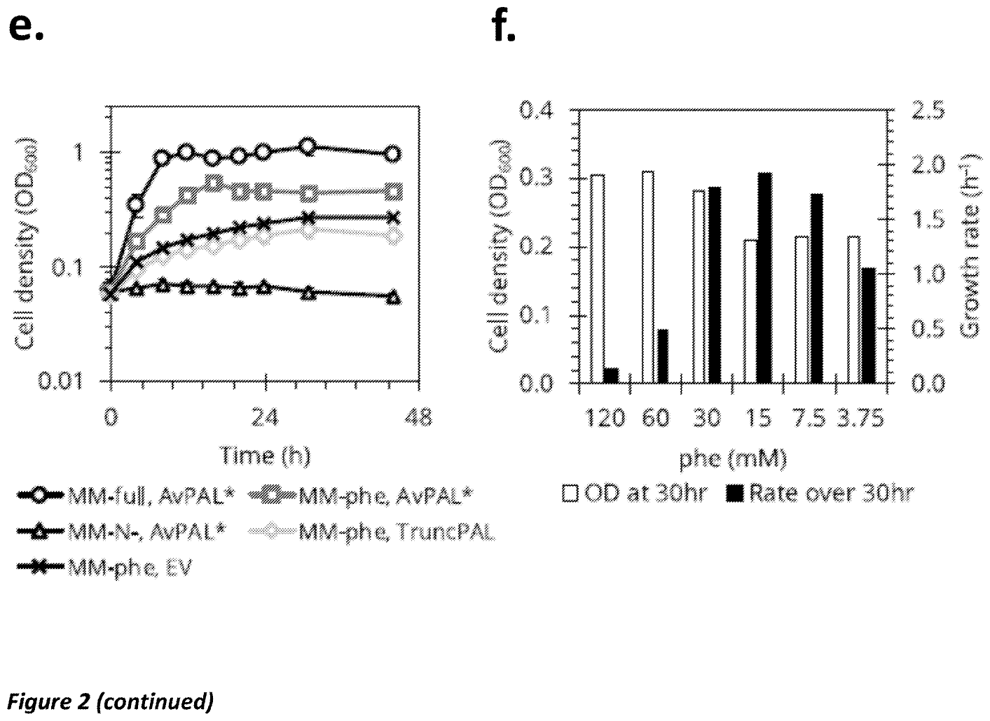

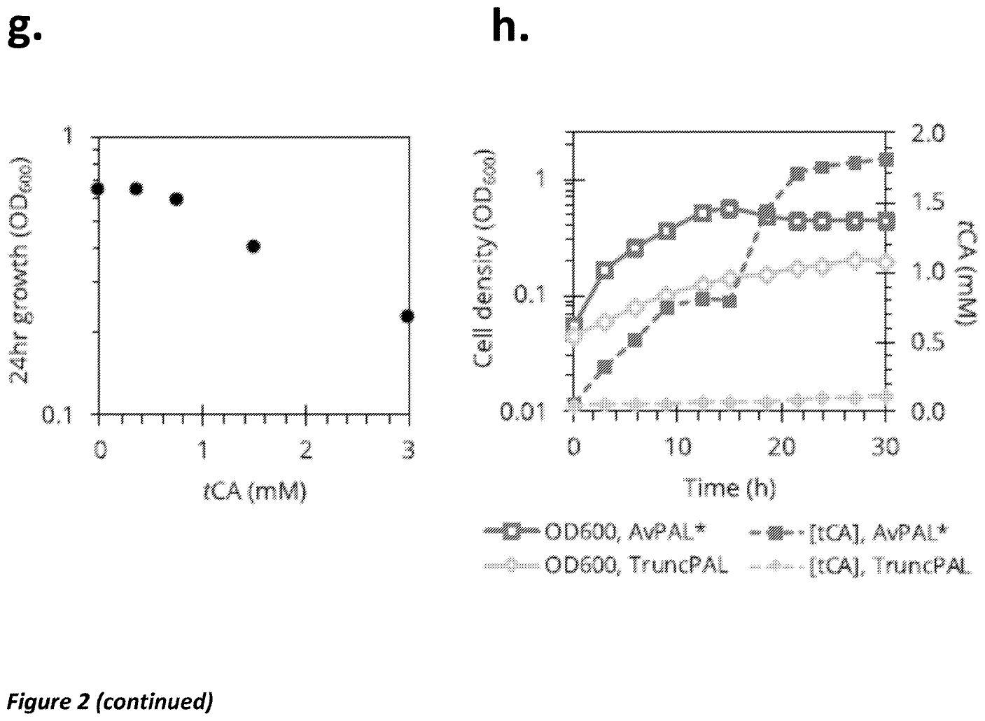

Subsequently, we optimized conditions to increase growth rate and shorten the lag by testing different media formulations (carbon source, pH, strain background, culture volume, phe concentration, and the presence of an additional nitrogen source) (FIG. 2a-d). We initially performed this optimization using MG1655, which grows poorly in minimal media because of inefficient pyrimidine utilization from a mutation in rph. Switching to a strain with a corrected allele (MG1655^{rph+}) shortened the lag phase by 18 h and culturing in glucose reduced the lag phase by another 24 h (FIG. 2e).

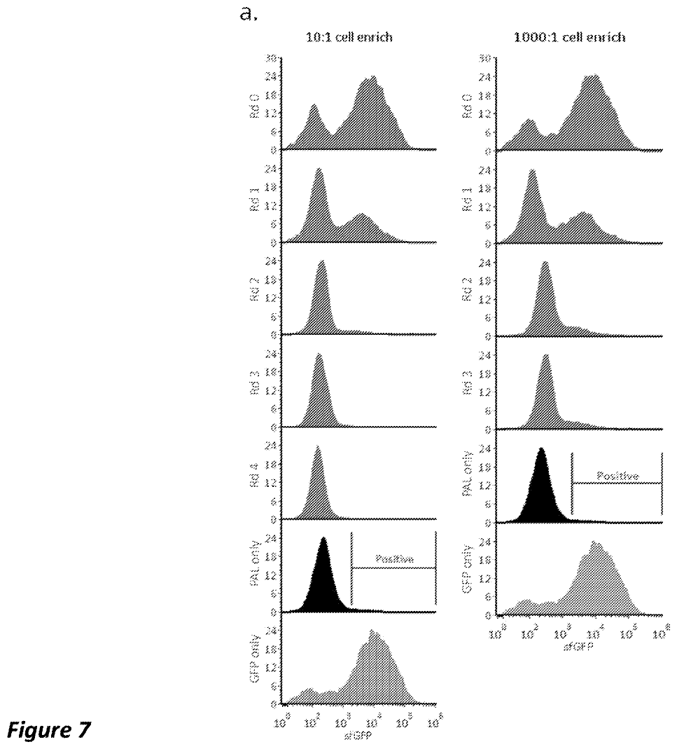

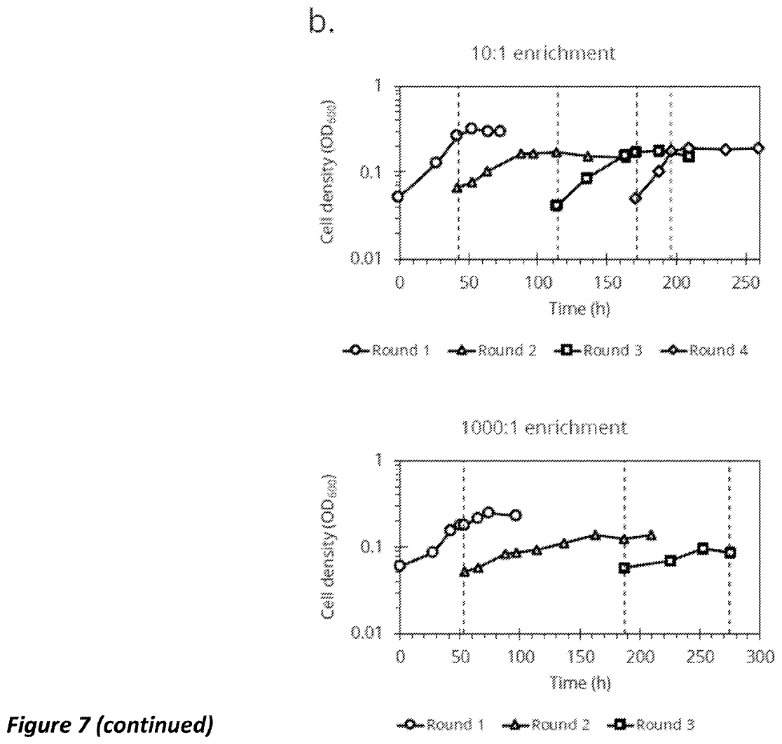

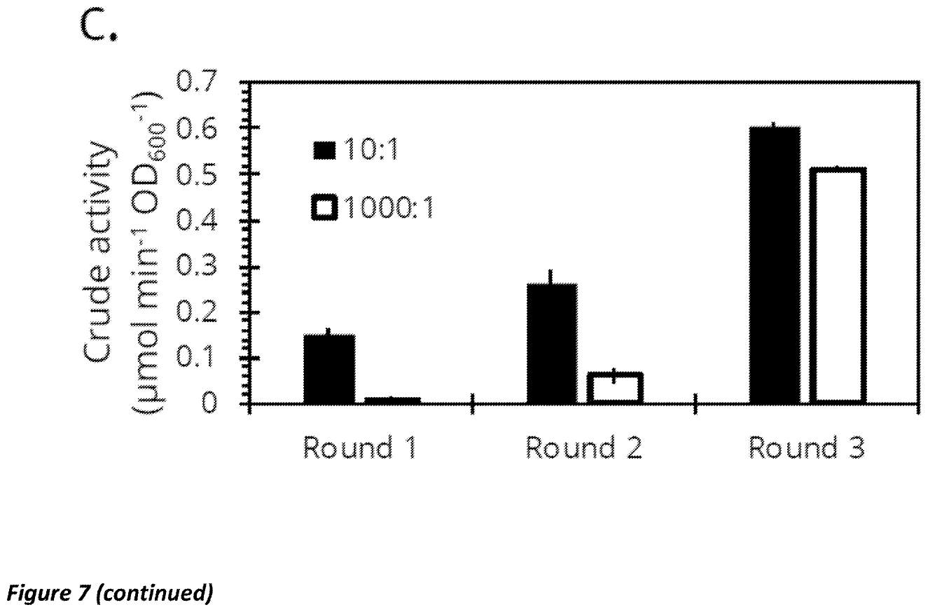

We found that not only was phe concentration important for optimal growth (FIG. 2a-d,f), but that trans-cinnamic acid (tCA) was toxic to cells. Cells grown in MM^{full,opt} showed impaired growth when supplemented with 1.5 mM tCA (FIG. 20. When grown in MM^{phe,opt} containing <30 mM phe, growth rate and biomass yield were reduced by low nitrogen availability. However, at phe >30 mM, tCA accumulated too quickly causing toxicity, and the cells not only experience a long lag but also quickly arrest growth (FIG. 2f-h). The final optimized conditions in FIG. 2h show that despite growth levelling out at OD₆₀₀ 0.3, the lag was virtually eliminated. Thus, we determined that subculturing the cells into fresh medium at OD₆₀₀ 0.2 would minimize tCA toxicity and basal growth—maximizing the difference between inactive and active PAL expressing cells. To validate enrichment occurs under these conditions, we created a mock library by transforming a 1:10 or 1:1000 mixture of AvPAL*-to-sfGFP-expressing. We measured cell fluorescence by flow-cytometry and observed decreasing fluorescence and increasing PAL activity at successive rounds of subculture in MM^{phe,opt} (FIG. 7).

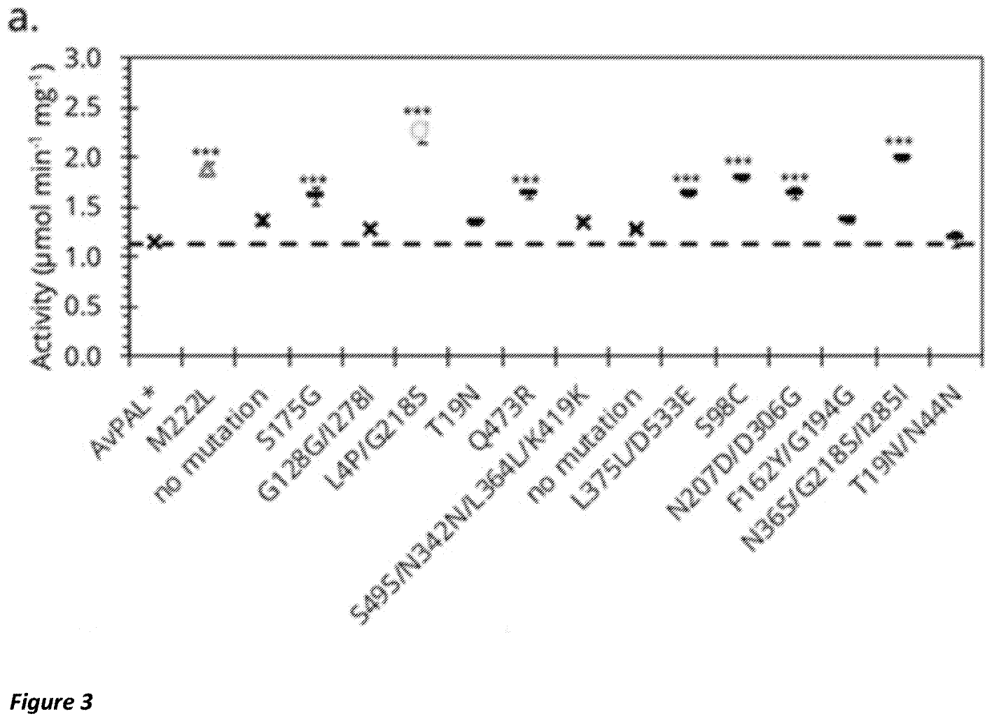

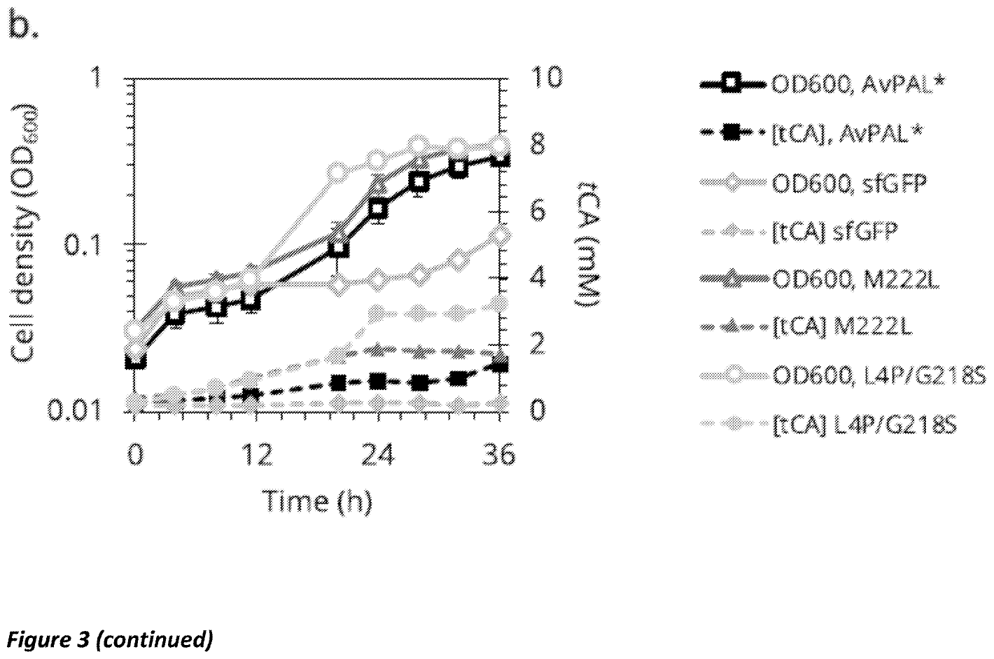

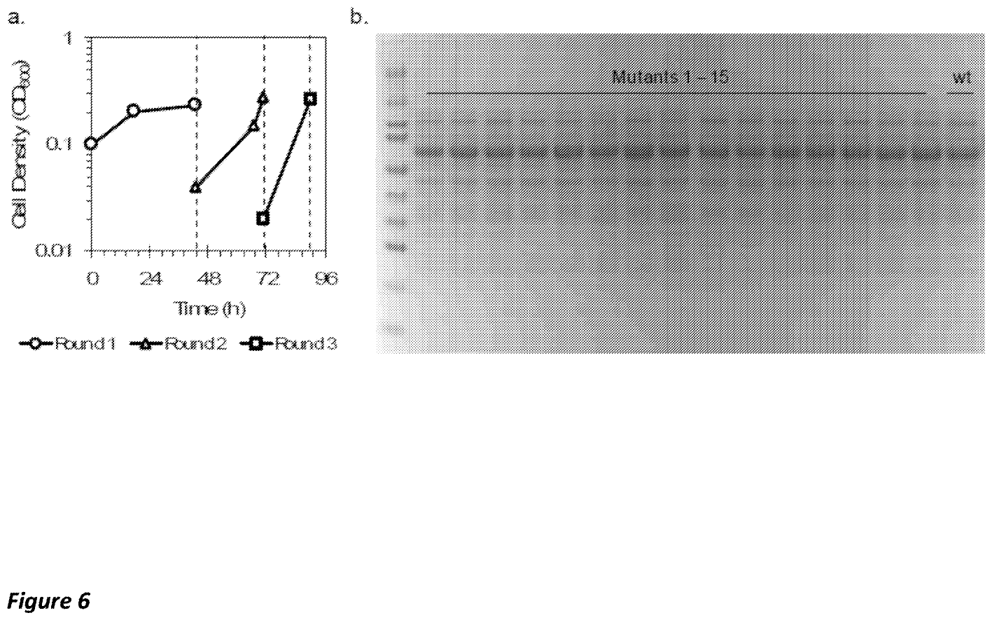

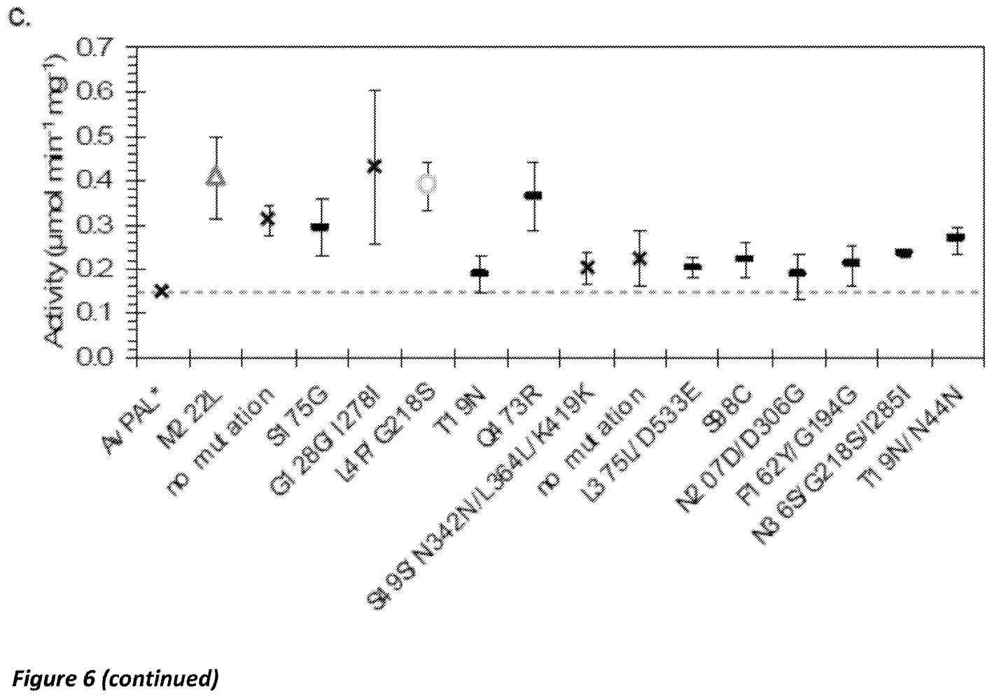

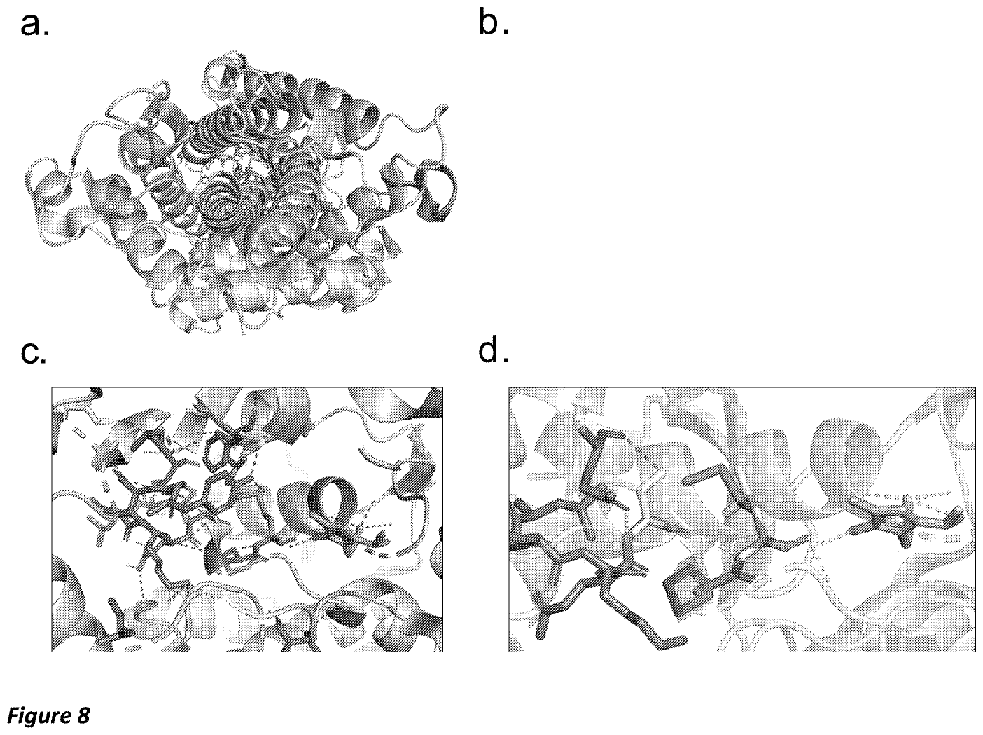

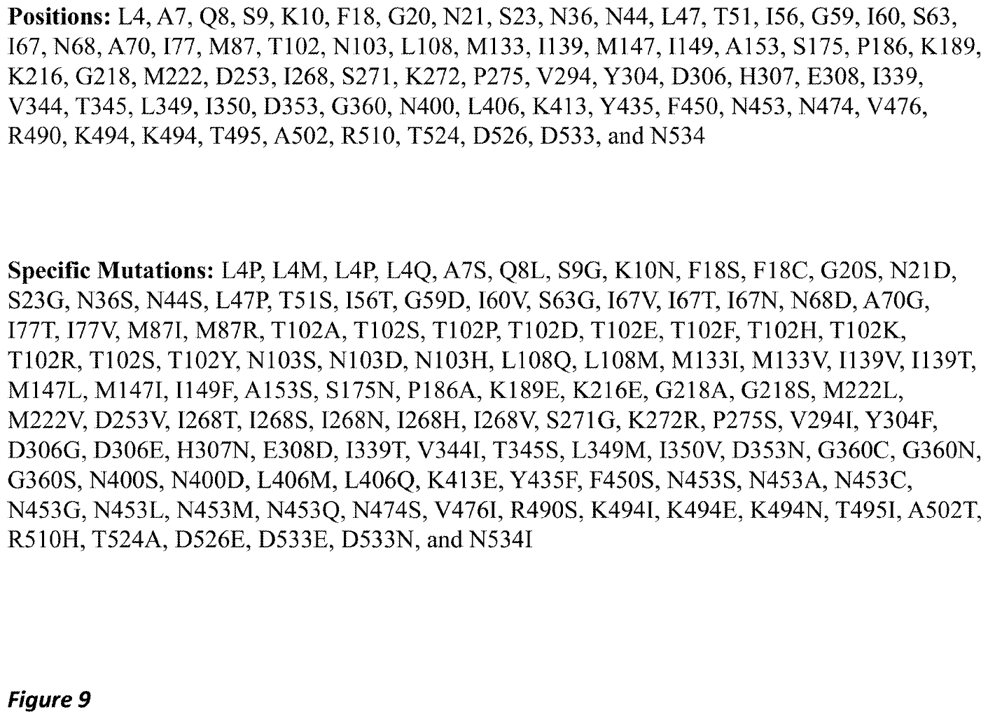

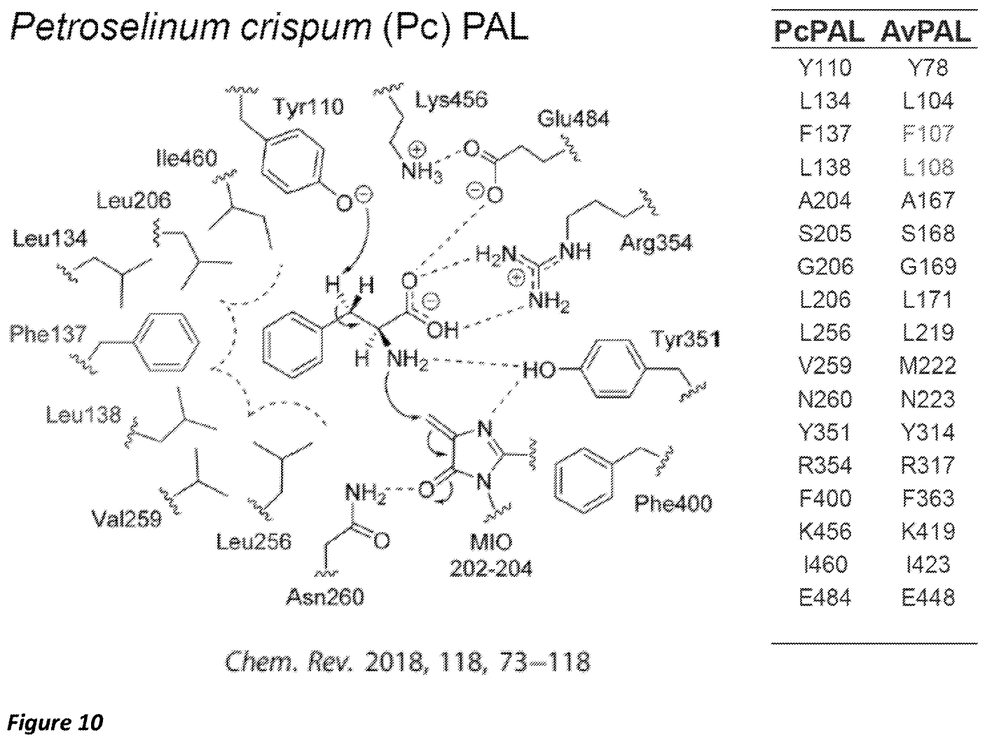

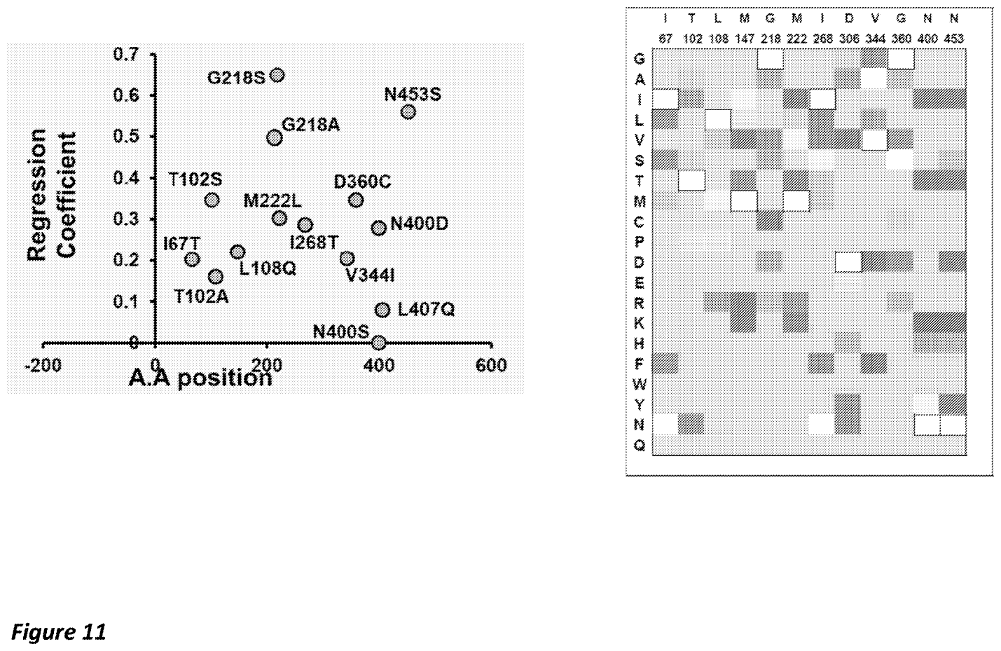

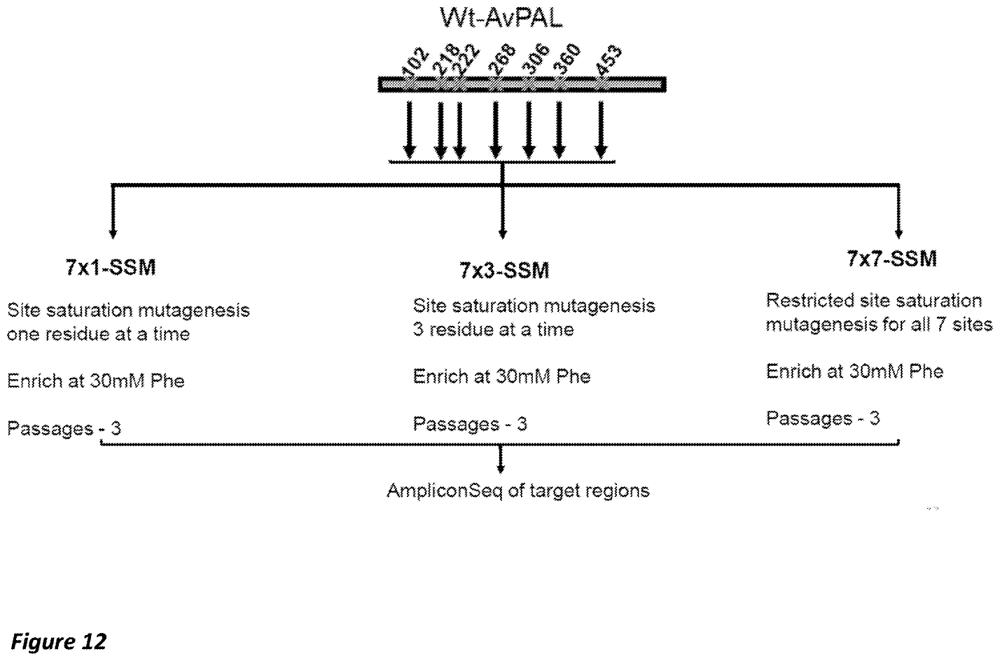

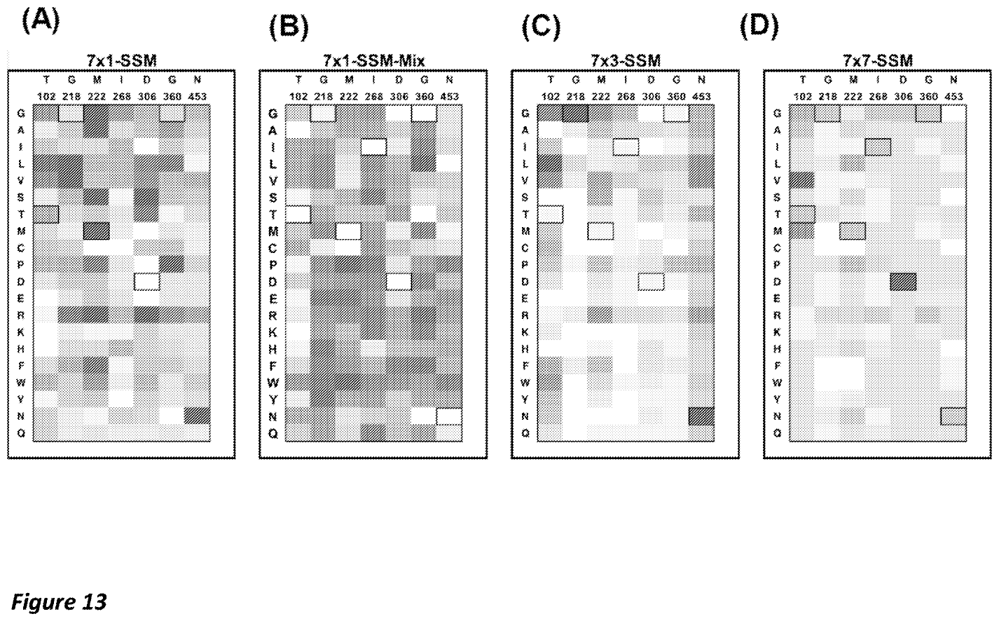

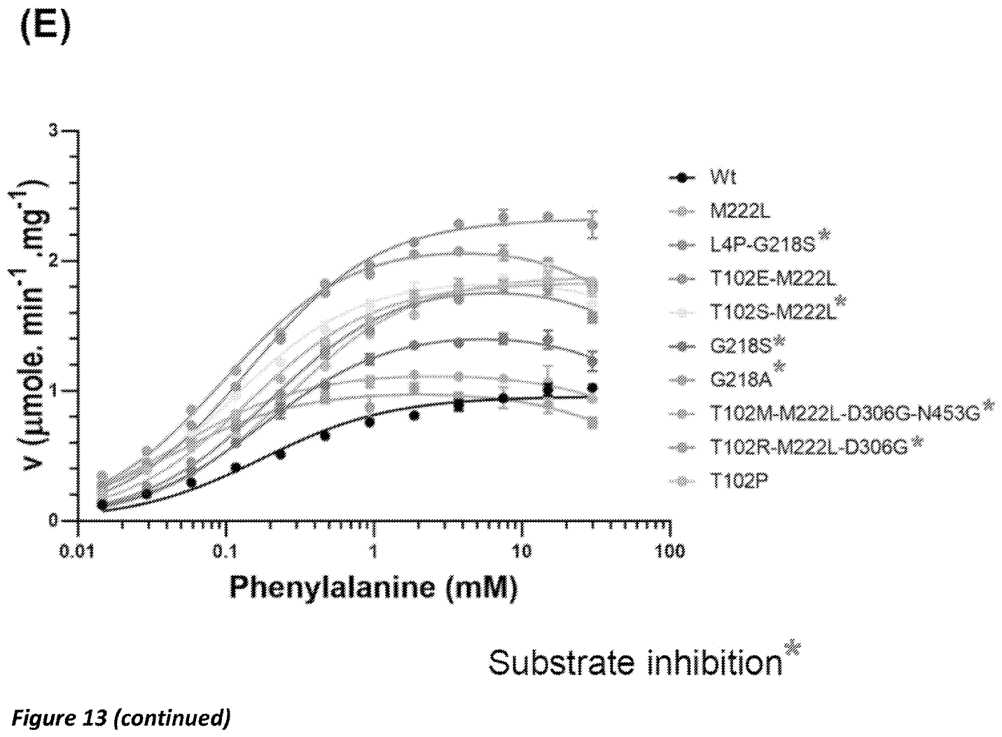

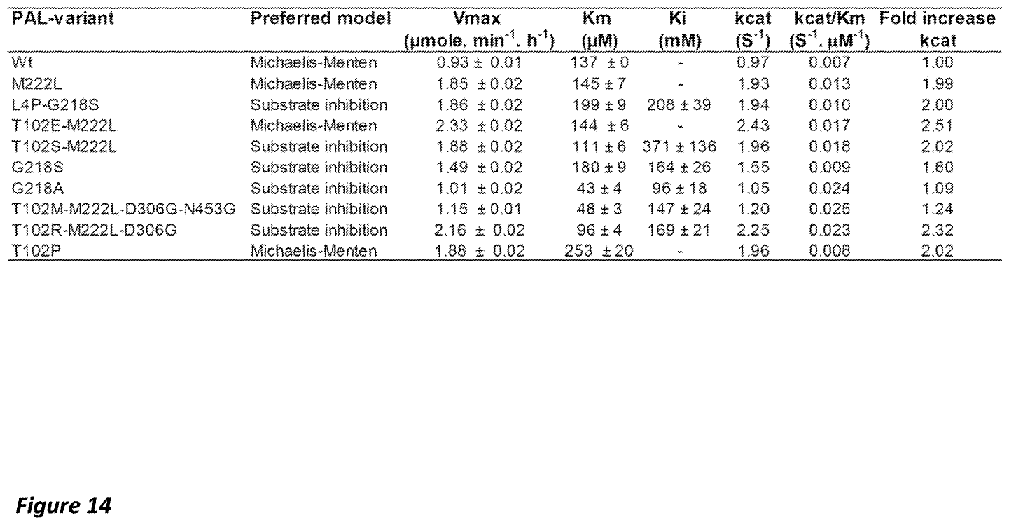

After finalizing the conditions to enrich active PAL, we created a mutant library of 10⁵ variants with an average error rate of 2.8 aa/protein. The entire library was grown in MM^{phe,opt} over three rounds, subculturing each time at OD₆₀₀ of 0.2 (FIG. 6). We subsequently plated the cells on non-selective LB medium, picked fifteen random colonies and screened their lysates (FIG. 7c) and purified protein (FIG. 3a) for PAL activity. Eight of the fifteen, including M222L and L4P/G218S, showed 1.5- to 2-fold higher activity with the other three showing similar activity to parental AvPAL* (FIG. 3a). Colonies with same sense or no mutation showed PAL activity similar to that of AvPAL*. This result suggests successful enrichment of higher activity mutants over lower/inactive mutants. E. coli expressing M222L and L4P/G218S mutants showed faster growth compared to AvPAL* in MM^{phe,opt}, with all attaining the same OD₆₀₀ at stationary phase (FIG. 3b). The greater differences in growth profiles at early growth stages between mutants (M222L and L4P/G218S) and parental AvPAL* is consistent with the enrichment strategy of subculturing at low OD₆₀₀. Furthermore, residues 218 and 222 are directly adjacent to the active site of AvPAL* and in close vicinity of the MIO-adduct. Comparing the crystal structure of AvPAL* to these mutants shows potential changes in hydrogen bonding within the active site (FIG. 8).

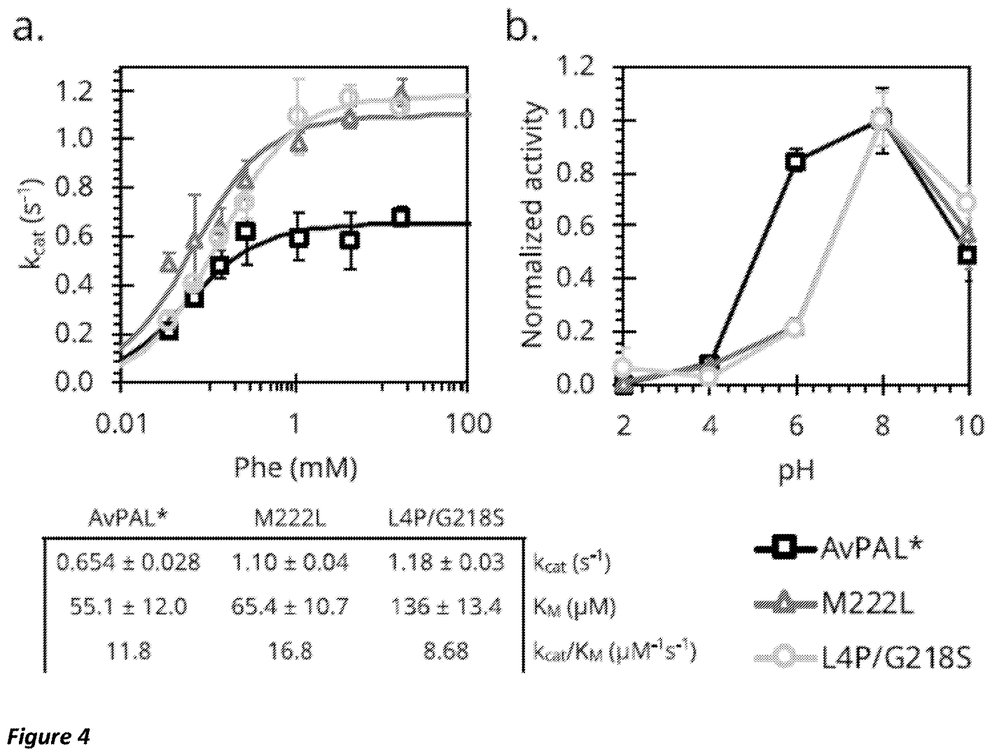

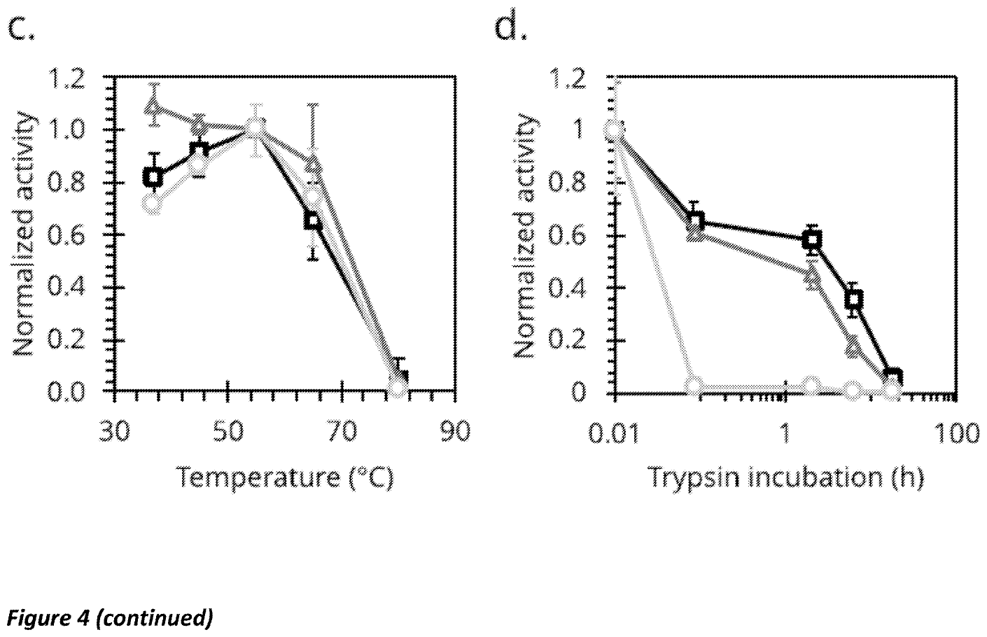

Previous studies with AvPAL* have demonstrated that kinetic parameters, pH optimum, thermal and proteolytic stabilities are relevant to therapeutic efficacy for PKU enzyme-replacement therapy. The k_{cat} of both the mutants was 70-80% higher than parental AvPAL* (FIG. 4a) whereas the K_{M} of M222L was similar to that of the parent and that of L4P/G218S was ˜2.5×higher. Overall, the M222L mutant showed improved catalytic efficiency compared to AvPAL*, while L4P/G218S mutant showed a trade-off between turnover frequency and substrate “affinity”. AvPAL* is reported to have a pH optimum in the range of 7.5-8.5²⁰ and we observed similar results for both the mutants (FIG. 4b), albeit with a slightly narrower optimal range. Temperature stability was assessed by subjecting the mutants to different temperatures for 1 h before measuring enzyme activity at optimal conditions (37° C., pH 7.4). The enzymes remained stable from 37° C. to 55° C. and began a modest decrease in relative activity at 65° C. before denaturing at 80° C. (FIG. 4c). The proteolytic stability was evaluated by incubating purified enzymes to trypsin. M222L was as trypsin-resistant as AvPAL* but L4P/G218S showed rapid loss of activity within five minutes (FIG. 4d).

Our results show that the catalytic properties of this class of enzymes, which are important for both industrial and biomedical applications, can be engineered using directed evolution. Further, the large sequence space we rapidly searched to identify mutations at residues previously unrecognized as functionally important, serves as evidence of this technique's strength. Since deamination activity serves as the foundation of technique, we offer this method as demonstration that may be applicable to other ALs as well. While the enzyme AvPAL* has specific implications as a therapy for PKU, several other ALs are under clinical investigation to treat disorders and biosynthesize industry chemicals. Our method could further direct AL temperature and pH stability or proteolytic resistance. The improvements in turnover rates observed here are unprecedented in the literature, either through rational or combinatorial methods, and has tremendous translation potential, especially for PKU.

Methods

Microbial strains, plasmids, and growth conditions. All Escherichia coli strains were cultured in lysogeny broth (LB) (VWR International, Randor, Pa.) at 37° C. with rotary shaking at 250 rpm. All media was solidified using 1.5% (w/v) agar (Teknova Inc, Hollister, Calif.). Minimal media (MM) conditions are described in the “Optimization of growth-coupled enrichment” section below. E. coli DH5a was used as a host for the construction of the expression vectors and cultured as above only supplemented with chloramphenicol (25 μg/mL) (RPI Corp). Initial expression in MM was performed in E. coli MG1655(DE3)^{ΔendA,ΔrecA} and later moved to E. coli MG1655^{rph+} for final experiments.

All cloning was performed in E. coli DH5a with reagents from New England Biolabs, Inc (Ipswich, Mass.). Preliminary expression experiments were conducted using the inducible pACYC-Duet1_AvPAL*, constructed by using the surrounding sites for restriction endonucleases NcoI and XhoI. For subsequent experiments requiring constitutive expression, the plasmid pBAV1k was used to express AvPAL*.

Enzyme activity assays. The activity of all AvPAL constructs was measured by production of tCA over time. Cultures were sonicated on ice using a Sonifier SFX 150 (Branson Ultrasonics, Danbury, Conn.) (2 s on; 10 s off; 4 min; 55%), and debris was separated from the lysate by centrifuging at 10,000×g for 10 min. Ten microliters of lysate were then mixed with 190 μL of pre-warmed 50 mM phe (Tokyo Chemical Industry, Portland, Oreg.) in phosphate buffered saline (PBS) (137 mM NaCl, 2.7 mM KCl, 10 mM Na₂HPO₄, 2 mM KH₂PO₄, pH 7.4) in a 96-well F-bottom UVStar (Greiner Bio-One, Kremsmünster, Austria) microtiter plate. Absorbance at 290 nm was measured every 30 s at 37° C. using a SpectraMax M3 (Molecular Devices) plate reader.

Each construct included a N-term His-tag used for immobilized metal affinity chromatography (IMAC) purification. Briefly, overnight cell cultures were sonicated in 3 mL Equilibration buffer (300 mM NaCl, 50 mM NaH₂PO₄, 10 mM imidazole, 15% (w/v) glycerol, pH 8.0). The lysate was loaded onto a prepared column with 2 mL TALON Metal Affinity Resin (Clontech Laboratories, Inc., Mountain View, Calif.). After being washed twice with 5 column volumes (CV) of Equilibration buffer, pure protein was then eluted off the column with 2.5 mL of Elution buffer (300 mM NaCl, 50 mM NaH₂PO₄, 500 mM imidazole, 15% (w/v) glycerol, pH 8.0), collecting 0.5 CV fractions until dry. Elution fractions showing clean protein bands on an SDS-PAGE were then dialyzed and concentrated in Storage buffer (20% (v/v) glycerol in PBS, pH 7.4) using a 10K MWCO Microsep Advance Centrifugal Device (Pall Corporation, Port Washington, N.Y.) as directed. Purified protein extracts were aliquoted and stored at −20° C., replacing lysate in subsequent characterization and activity assays. Protein concentration was measured by Bradford method using bovine serum albumin (BSA) as the standard.

AvPAL library creation. Random mutagenesis libraries were created using two rounds of error prone PCR, with the amplicon of the first reaction serving as the template DNA for the second. Each reaction contained 1× Standard Taq reaction buffer (New England Biolabs, Inc.), 5 mM MgCl₂, 0.15 mM MnCl₂, 0.2 mM dATP, 0.2 mM dGTP, 1 mM dCTP, 1 mM dTTP, 0.4 μM each primer, 0.4 ng/μL template DNA, and 0.05 U/ml Taq DNA polymerase. The reactions were amplified using the following PCR cycle conditions: 95° C. denaturation, 1 min; 16 cycles of 95° C. denaturation, 30 s; 46° C. annealing, 45 s; and 68° C. extension, 2 min, followed by 68° C. extension for 5 min. The target vector, pBAV1k was amplified separately using Phusion PCR, and the two were combined using Gibson assembly. The reaction was purified with a E.Z.N.A. Cycle Pure Kit (Omega) before being transformed by electroporation into E. coli MG1655^{rph+}.

Optimization of growth-coupled enrichment. Growth was measured by seeding cultures at OD₆₀₀ 0.05 and monitoring cell density over time. Initial experiments used a base nitrogen-deficient minimal media (MM^{N-}) (33.7 mM Na₂HPO₄, 22 mM KH₂PO₄, 8.55 mM NaCl, 1 mM MgSO₄, 0.1 mM CaCl₂), 10 μM FeSO₄, 0.4% (v/v) glycerol, 10 μg/mL thiamine, 20 μM IPTG, 12.5 μg/mL chloramphenicol, pH 7.4) that was supplemented with 9.35 mM phe (MM^{phe,init}) or 9.35 mM NH₄Cl) Variable conditions were changed across the parameters outlined in FIG. 2, as well as moving to a more favorable strain for growth in minimal media. This resulted in a final MM^{N-,opt} (137 mM NaCl, 2.7 mM KCl, 10 mM Na₂HPO₄, 2 mM KH₂PO₄, 2 mM MgSO₄, 1× Trace Metals (Teknova, Inc.), 0.2% (v/v) glucose, 10 μg/mL thiamine, 12.5 μg/mL chloramphenicol, pH 7.4) supplemented with 30 mM phe (mm^{phe,opt}) or 9.35 mM NH₄Cl (MM^{full,opt}). To enrich the active population of the AvPAL library, cells were subcultured into fresh MM^{phe,opt} once they reached OD₆₀₀ 0.2.

Flow cytometry. Plasmids, both with a pBAV1k backbone, expressing sfGFP or AvPAL* were mixed in a 1000:1 or 10:1 ratio as a mock mutant library and transformed by electroporation into E. coli MG1655^{rph+}. Cells were recovered for 1 h before being washed and seeded in 5 mL of selective media as prepared above. Cell density was measured over time until reaching OD₆₀₀ 0.2, when the cells were subcultured to OD₆₀₀ 0.05 for the next round of enrichment. Cells were also plated at each subculture for PCR amplification to confirm the presence of either sfGFP or AvPAL*. Cells at each point of subculture were also diluted to OD₆₀₀ 0.05 for flow cytometry analysis. A minimum of 10,000 events were collected using a blue laser on an Attune N×T flow cytometer (Life Technologies, Carlsbad, Calif.). Fluorescence of sfGFP was detected on the BL1-H channel with 488 nm excitation, and loss of fluorescence was revealed as a measure of active AvPAL* enrichment.

Enzyme kinetics. AvPAL* and selected mutants were purified as described above. The activity of 0.1 μg of protein was measured by the production of tCA over 10 min by recording the absorbance of the reaction mix at 290 nm. Phe was added at varying concentrations from 35 μM to 17.5 mM in PBS, pH 7.4 (PBS) at 37° C. to begin the reaction. A Michaelis-Menten curve was fit in GraphPad Prism software using the initial rate at each phe concentration.

pH profile. The optimal pH of AvPAL* and selected mutants was determined by performing the enzyme activity described above. A 35 mM phe solution was buffered across a pH range (2 to 10) using phosphate-citrate buffer, prepared by varying concentrations of Na₂HPO₄ and citric acid. Total 0.2 μg protein was used to carry out the activity reaction in 200 μL at 37° C.

Temperature stability. The effect of temperature on the stability of AvPAL* and selected mutants was determined by incubating the protein in PBS, pH 7.4 at temperatures ranging from 37° C. to 80° C. for 1 hour followed by measuring the enzyme activity. Each enzyme reaction was carried out using 1 μg of PAL protein and 35 mM phe as substrate in a total reaction volume of 200 μL at 37° C.

Proteolytic stability. The proteolytic stability was evaluated by subjecting AvPAL* and selected mutants to a catalytic amount of trypsin as previously described¹. Briefly, 100 μg/mL AvPAL enzyme was subjected to trypsin (40 μg/mL) (MilliporeSigma, Burlington, Mass.) in PBS at 37° C. Enzyme activity of 1 μg of protein was then measured as described above.

### REFERENCES

[1] H. A. Cooke, C. v. Christianson, S. D. Bruner, Current Opinion in Chemical Biology 2009, 13, 453-461.
[2] H. Lee, B. G. Kim, M. Kim, J. H. Ahn, Journal of Microbiology and Biotechnology 2015, 25, 1442-1448.
[3] K. D. Walker, K. Klettke, T. Akiyama, R. Croteau, Journal of Biological Chemistry 2004, 279, 53947-53954.
[4] J. An, M. Zhang, Q. Lu, Journal of Food Engineering 2007, 78, 340-344.
[5] S. Y. Shin, S. M. Jung, M. D. Kim, N. S. Han, J. H. Seo, Enzyme and Microbial Technology 2012, 51, 211-216.
[6] R. E. Hoagland, Weed Technology 1996, 10, 651-674.
[7] R. McKenna, D. R. Nielsen, Food, Pharmaceutical and Bioengineering Division—Core Programming Topic at the 2011 AIChE Annual Meeting 2011, 1, 340-350.
[8] S. Verhoef, H. J. Ruijssenaars, J. A. M. de Bont, J. Wery, Journal of Biotechnology 2007, 132, 49-56.
[9] S. Verhoef, N. Wierckx, R. G. M. Westerhof, J. H. de Winde, H. J. Ruijssenaars, Applied and Environmental Microbiology 2009, 75, 931-936.
[10] H. Jiang, K. v. Wood, J. A. Morgan, Applied and Environmental Microbiology 2005, 71, 2962-2969.
[11] J. Wu, G. Du, J. Zhou, J. Chen, Metabolic Engineering 2013, 16,
[12] N. il Park, H. Xu, X. Li, Y. S. Kim, M. Y. Lee, S. U. Park, Process Biochemistry 2012, 47, 2575-2580.
[13] T. Hydery, V. A. Coppenrath, Drug Target Insights 2019, 13, DOI 10.1177/1177392819857089.
[14] M. T. Castañeda, O. Adachi, R. A. Hours, Journal of Industrial Microbiology and Biotechnology 2015, 42, 1299-1307.
[15] U.S. Pat. No. 7,560,263B2, 2009.
[16] R. Shen, R. R. Fritz, C. W. Abell, Cancer Research 1977, 37, 1051-1056.
[17] C. Chesters, M. Wilding, M. Goodall, J. Micklefield, Angewandte Chemie—International Edition 2012, 51, 4344-4348.
[18] S. M. Bell, D. J. Wendt, Y. Zhang, T. W. Taylor, S. Long, L. Tsuruda, B. Zhao, P. Laipis, P. A. Fitzpatrick, PLOS ONE 2017, 12, e0173269.
[19] F. Zhang, N. Huang, L. Zhou, W. Cui, Z. Liu, L. Zhu, Y. Liu, Z. Zhou, Applied Biochemistry and Biotechnology 2017, 183, 699-711.
[20] L. Wang, A. Gamez, H. Archer, E. E. Abola, C. N. Sarkissian, P. Fitzpatrick, D. Wendt, Y. Zhang, M. Vellard, J. Bliesath, et al., Journal of Molecular Biology 2008, 380, 623-635.
[21] Lovelock S L and Turner N J, Bioorganic and Medicinal Chemistry, 2014, 22, 5555-5557.
[22] G. v. Louie, M. E. Bowman, M. C. C. Moffitt, T. J. Baiga, B. S. S. Moore, J. P. Noel, Chemistry and Biology 2006, 13, 1327-1338.
[23] L. Xiang, B. S. Moore, Journal of Bacteriology 2005, 187, 4286-4289.
[24] S. Bartsch, G. G. Wybenga, M. Jansen, M. M. Heberling, B. Wu, B. W. Dijkstra, D. B. Janssen, ChemCatChem 2013, 5, 1797-1802.
[25] R. Wohlgemuth, Current Opinion in Microbiology 2010, 13, 283-292.
[26] B. Wu, W. Szymanski, P. Wietzes, S. de Wildeman, G. J. Poelarends, B. L. Feringa, D. B. Janssen, ChemBioChem 2009, 10, 338-344.
[27] N. J. Turner, Current Opinion in Chemical Biology 2011, 15, 234-240.
[28] E. E. Wrenbeck, L. R. Azouz, T. A. Whitehead, Nature Communications 2017, 8, 1-10.
[29] L. K. Flachbart, S. Sokolowsky, J. Marienhagen, ACS Synthetic Biology 2019, 8, 1847-1857.
[30] L. J. Reitzer, in Escherichia Coli and Salmonella: Cellular and Molecular Biology (Ed.: F. C. Neidhardt), American Society for Microbiology, Washington, D.C., 1996, pp. 380-390.
[31] D. H. Gelfand, R. A. Steinberg, Journal of Bacteriology 1977, 130, 429-440.
[32] G. I. Guzmán, J. Utrilla, S. Nurk, E. Brunk, J. M. Monk, A. Ebrahim, B. O. Palsson, A. M. Feist, Proceedings of the National Academy of Sciences 2015, 112, 929-934.
[33] K. A. J. F. Jensen, Journal of Bacteriology 1993, 175, 3401-3407.
[34] L. Wang, A. Gamez, H. Archer, E. E. Abola, C. N. Sarkissian, P. Fitzpatrick, D. Wendt, Y. Zhang, M. Vellard, J. Bliesath, S. M. Bell, J. F. Lemontt, C. R. Scriver and R. C. Stevens, Journal of Molecular Biology, 2008, 380, 623-635.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A variant phenylalanine ammonia-lyase (PAL) enzyme of Anabaena variabilis (SEQ ID NO:1) comprising amino acid substitutions that comprise: (i) C503S and C565S (SEQ ID NO:2); and one or more of (ii) L4P, L4M, L4P, L4Q, A7S, Q8L, 59G, K10N, F185, F18C, G20S, N21D, S23G, N36S, N445, L47P, T51S, I56T, G59D, I60V, S63G, I67V, I67T, I67N, N68D, A70G, I77T, I77V, M87I, M87R, T102A, T102S, T102P, T102D, T102E, T102F, T102H, T102K, T102R, T102S, T102Y, N103S, N103D, N103H, L108Q, L108M, M133I, M133V, I139V, I139T, M147L, M147I, I149F, A1535, S175N, P186A, K189E, K216E, G218A, G2185, M222L, M222V, D253V, I268T, I268S, I268N, I268H, I268V, S271G, K272R, P275S, V294I, Y304F, D306G, D306E, H307N, E308D, I339T, V344I, T345S, L349M, I350V, D353N, G360C, G360N, G360S, N400S, N400D, L406M, L406Q, K413E, Y435F, F450S, N453S, N453A, N453C, N453G, N453L, N453M, N453Q, N474S, V476I, R490S, K494I, K494E, K494N, T495I, A502T, R510H, T524A, D526E, D533E, D533N, and N534I.

2. The variant PAL enzyme of claim 1, wherein the variant has a k_{cat} with respect to producing trans-cinnamic acid from phenylalanine that is higher than wild-type Anabaena variabilis (SEQ ID NO:1) or a variant PAL enzyme of Anabaena variabilis (SEQ ID NO:1) comprising amino acid substitutions that comprise C503S and C565S (SEQ ID NO:2).

3. The variant PAL enzyme of claim 1, wherein the variant exhibits the same stability or higher stability at a temperature of at least 37° C., 45° C., 45° C., 50° C., 55° C., 60° C., or 65° C. than wild-type Anabaena variabilis (SEQ ID NO:1) or a variant PAL enzyme of Anabaena variabilis (SEQ ID NO:1) comprising amino acid substitutions that comprise C503S and C565S (SEQ ID NO:2).

4. A conjugate comprising the variant PAL enzyme of claim 1 conjugated to a polyethylene glycol (PEG) polymer.

5. A pharmaceutical composition comprising; (i) the variant PAL enzyme of claim 1 or a conjugate comprising the variant PAL enzyme conjugated to a polyethylene glycol (PEG) polymer and (ii) a suitable pharmaceutical carrier.

6. A polynucleotide encoding the variant PAL enzyme of claim 1.

7. The polynucleotide of claim 6, wherein the polynucleotide is codon-optimized for expression of the variant PAL enzyme in Escherichia coli.

8. The polynucleotide of claim 6, wherein the polynucleotide is codon-optimized for expression of the variant PAL enzyme in human cells.

9. An expression vector comprising a promoter operably linked to the polynucleotide of claim 6.

10. A modified cell comprising: a polynucleotide encoding the variant PAL enzyme of claim 1; and/or an expression vector comprising a promoter operably linked to the polynucleotide encoding the variant PAL enzyme, optionally wherein the cell is a modified Escherichia coli cell or a modified human cell.

11. A method for preparing a variant PAL enzyme, the method comprising culturing the modified cell of claim 10 in culture media to express the variant PAL enzyme and isolating the variant PAL enzyme from the modified cell and/or from the culture media.

12. A method for treating a disease or disorder in a subject in need thereof, wherein the disease or disorder is characterized by elevated blood levels of phenylalanine, the method comprising administering to the subject the variant PAL enzyme of claim 1, a conjugate comprising the variant PAL enzyme conjugated to a polyethylene glycol (PEG) polymer, or a pharmaceutical composition comprising the variant PAL enzyme or the conjugate.

13. The method of claim 12, wherein the subject is administered the variant PAL enzyme, the conjugate, or the pharmaceutical composition subcutaneously.

14. The method of claim 12, wherein the disease or disorder is phenylketonuria (PKU).

15. A method for obtaining a variant of a PAL enzyme; the method comprising one or more of the following steps: (i) transforming cells that cannot utilize phenylalanine to obtain nitrogen with a library of expression vectors that encode and express variants of PAL enzymes in the transformed cells; (ii) culturing the transformed cells in a minimal media that is supplemented with phenylalanine; (iii) selecting transformed cells that grow in the minimal media that is supplemented with phenylalanine; (iv) determining the sequence of the encoded PAL enzyme of a transformed cell that grows in the minimal media that is supplemented with phenylalanine, thereby obtaining the variant of the PAL enzyme.

16. The method of claim 15, wherein the variant of PAL enzyme is a variant of Anabaena variabilis PAL enzyme.

17. The method of claim 15, wherein the transformed cells are transformed Escherichia coli and the expression vectors have been codon-optimized for expression of the variant of the PAL enzyme in Escherichia coli.

18. The method of claim 15, wherein the minimal media is supplemented with glucose, optionally at a concentration of 0.1-0.3 (v/v)).

19. The method of claim 15, wherein the minimal media does not comprise glycerol.

20. The method of claim 15, wherein the minimal media is supplemented with phenylalanine at a concentration of 20-40 mM.

21. The method of claim 15, wherein culturing comprises subculturing the transformed cells by removing the cells from the culture media after the culture media reaches an OD₆₀₀ of at least about 1.8-2.2 and placing the transformed cells into fresh minimal media supplemented with phenylalanine.
